(19) United States
(12) Patent Application Publication
(10) Pub. No.: US 2007/0134332 A1
(43) Pub. Date: Jun. 14, 2007

(54) Polymer particles for delivery of macromolecules and methods of use

(75) Inventors: William D. Turnell, San Diego, CA (US); Geoffrey C. Landis, Carlsbad, CA (US); Zaza D. Gomurashvili, La Jolla, CA (US); Hong Li, San Diego, CA (US); Kristin DeFife, San Diego, CA (US); Vassil P. Vassilev, San Diego, CA (US); Yumin Yuan, San Diego, CA (US)

Correspondence Address: DLA PIPER US LLP, 4365 EXECUTIVE DRIVE, SUITE 1100, SAN DIEGO, CA, 92121-2133, US

(73) Assignee: MediVas, LLC, San Diego, CA (US)

(21) Appl. No.: 11/603,660

(22) Filed: Nov. 21, 2006

Related U.S. Application Data
(60) Provisional application No. 60/796,067, filed on Apr. 27, 2006.
(60) Provisional application No. 60/738,769, filed on Nov. 21, 2005.

(51) Int. Cl. A61K 48/00; A61K 38/28; C08F 20/00; A61K 9/14

(52) U.S. Cl. 424/486; 514/3; 514/4; 525/440; 514/44

(57) ABSTRACT

The present invention provides biodegradable polymer particle delivery compositions for delivery of macromolecular biologics, for example in crystal form, based on polymers, such as polyester amide (PEA), polyester urethane (PEUR), and polyester urea (PEU) polymers, which contain amino acids in the polymer. The polymer particle delivery compositions can be formulated either as a liquid dispersion or a lyophilized powder of polymer particles containing bound water molecules with the macromolecular biologics, for example insulin, dispersed in the particles. Bioactive agents, such as drugs, polypeptides, and polynucleotides can also be delivered by using particles sized for local, oral, mucosal or circulatory delivery. Methods of delivering a macromolecular biologic with substantial native activity to a subject, for example orally, are also included.

This application relies for priority under 35 U.S.C. § 119(e) on U.S. Ser. No. 60/796,067, filed Apr. 27, 2006 and U.S. Ser. No. 60/738,769, filed Nov. 21, 2005, which are incorporated herein by reference.

### FIELD OF THE INVENTION

The invention relates, in general, to drug delivery systems and, in particular, to polymer particle delivery compositions that can deliver a variety of different macromolecules in a time release fashion.

### BACKGROUND INFORMATION

Biologic macromolecules constitute a large and important class of therapeutic compounds. Such macromolecules are composed of one or more polymeric chains, forming a three-dimensional structure held together by non-covalent forces, both hydrophobic and ionic, such as is observed in native or synthetically produced proteins and polynucleic acids. The majority of these macromolecules have to be administered by injection or via a catheter to avoid the destruction of their three-dimensional structure upon which their biological activity depends. There are many barriers in vivo preventing the delivery of such biologic macromolecules to their target tissue via routes of administration other than by injection or via a catheter. Oral, rectal, vaginal and intra-nasal routes represent many challenges to safe delivery, including changes in pH and the action of hydrolase enzymes. In addition to the rapid destruction of biologic macromolecules by hydrolases, lack of bio-adhesion and bio-absorption at tissue surfaces can also contribute to the reduction of pharmacological efficacy of such macromolecules at the targeted tissue.

Many proteins and polypeptides are potentially therapeutic macromolecules that, in general, have prohibitively short half-lives when administered into biological milieu. Attempts to overcome these drawbacks have included the encapsulation of these biologics within bio-degradable formulations: either gels or particles made from natural polymers, such as carbohydrate hydrogels, or synthetic polymers, such as polyesters (e.g., PLGA). Release of the non-conjugated macromolecules from formulations of these types is controlled by a combination of diffusion and bio-erosion mechanisms due to the nature of the polymer itself.

To increase half-life, bio-adhesion, or tissue targeting, the biologic has been derivatized by covalent attachment to polymeric carrier molecules. For example, covalent attachment of carbohydrate or peptide chains to the biologic has been used for such purposes. Similarly, synthetic polymers, such as poly(ethylene glycol) (PEG) and methacrylates, have also been attached to biologics to extend half-life and increase bioadhesion. However, such synthetic polymers can have the disadvantage of limited natural bio-degradation, with the result that clearance from the body relies upon elution from tissues without full bio-degradation into smaller, component parts.

Unlike organic drug-like molecules and small biologics, such as short peptides, the activity in vivo of biologic macromolecules, and in particular of proteins, depends upon the constancy of their three-dimensional structure. The spatial, conformational fold of the macromolecular chain is held together by the concerted action of forces, each of which is far weaker than the covalent bonds of the macromolecular chain itself. All of these non-covalent forces are fundamentally electronic in nature: electrostatic ionic forces (including hydrogen bonding) or electrodynamic dispersion forces (short range hydrophobicity).

Open formulations, such as hydrogels, work to preserve therapeutic function by allowing the biologic molecules to bathe in a natural aqueous milieu. Extensive direct and water-bridged hydrogen bonding between the gel polymer and the biologic, in some cases coupled with local hydrophobic interactions, limits release of the biologic by diffusion through the gel. However, in many cases such open formulations allow ingress of degrading enzymes, which can infiltrate through the enzyme-sized pores of the gel, presenting an inherent problem for the delivery of biologic macromolecules with native activity.

Greater protection has been provided to the macromolecular biologic by hydrophobic polymers, which present a denser structure for the matrixing or encapsulation of macromolecular biologics. However, as hydrophobic polymers repel water, such synthetic polymer formulations have limited capacity for molecular interactions that help to preserve the native, folded state, and hence native activity, of the biologic. For example, the hydrophobic polyesters (e.g. PLGA) possess only limited ionic bonding capacity. In particular, polyesters lack hydrogen bond donors. Similarly, methacrylates are hydrophobic and must be extensively derivatized to introduce other, non-covalent bonding capacities. Moreover, most synthetic hydrophobic polymers have poor bio-erosion properties, or degrade via water/acid hydrolysis, resulting in degradation products that can modify the macromolecular biologic whose protection is being sought.

Delivery of oral insulin has been a primary goal of delivery technologies. For example, liposomes have been used to deliver insulin through the intestine mucosa, but have demonstrated some instability in the gut. Polymeric formulations have been developed to deliver insulin across the gut wall but the release of insulin is considered to be slow for the preprandial delivery of insulin. To overcome this problem, unnatural permeation enhancers, exogenous molecules that enhance the absorption of molecules through the gut wall, have also been used to enhance the absorption of insulin, but undesirable side effects in the gut have been recorded. For example, certain surfactants, which increase absorption, make holes in the gut so the subject becomes more susceptible to diseases and bowel irritations.

Chemists, biochemists, and chemical engineers are all looking beyond traditional polymer networks to find other innovative drug transport systems. Thus, there is still a need in the art for new and better polymer particle delivery compositions for controlled delivery of a variety of different types of macromolecular biologics.

### SUMMARY OF THE INVENTION

The present invention is based on the premise that amino acid-based PEAs, PEURs, and PEUs are biodegradable, synthetic polymers in which amino acid residues are linked together by short hydrocarbon chains derived from diols and di-acids, and can be used to form polymer particle delivery compositions for delivery of natural or man-made structurally intact macromolecular biologics. It is believed that the hydrophobic segments in PEA, PEUR and PEU containing polymers slow down the rate of bio-degradation of the polymer compared with that of proteins, probably by the repulsion of bulk water. As a consequence, the macromolecular biologics dispersed in the polymer are delivered in a consistent and reliable manner by biodegradation of the polymer.

The short hydrocarbon chains present in such polymers provide localized hydrophobic segments that act in concert with ionic regions provided by the amino acid residues to promote ionic bonding capacity, especially by providing hydrogen bond donors. The use of different lengths of hydrocarbon chains and different amino acids in the PEA, PEUR and PEU polymers generates variations that can be employed to optimize interactions between the polymer and the macromolecular biologic dispersed therein, enhancing stabilization of the macromolecular biologic. Thus, these bio-degradable polymers can be synthesized so as to possess non-covalent bonding capacities similar to those of natural macromolecular biologics, including proteins.

In one embodiment, the invention provides a polymer particle delivery composition in which at least one macromolecular biologic is dispersed in a biodegradable polymer, wherein the polymer comprises at least one or a blend of the following: a poly(ester amide) (PEA) having a chemical formula described by structural formula (I), 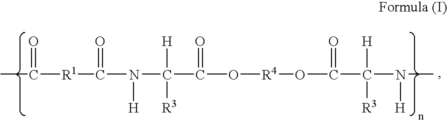
wherein n ranges from about 5 to about 150; R¹ is independently selected from residues of α,ω-bis (ohm or p 4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, or (C₂-C₂₀) alkenylene; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; and R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol, bicyclic-fragments of 1,4:3,6-dianhydrohexitols of structural formula (II), and combinations thereof; 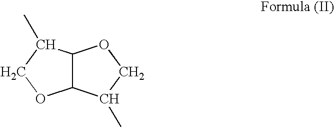
or a PEA having a chemical formula described by structural formula III: 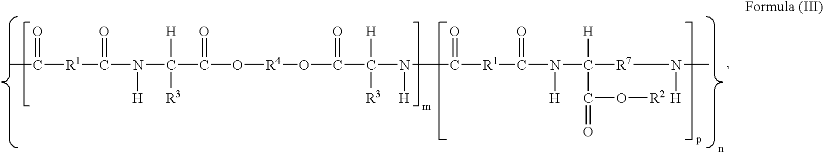
wherein n ranges from about 5 to about 150, m ranges about 0.1 to 0.9: p ranges from about 0.9 to 0.1; wherein R¹ is independently selected from residues of α,ω-bis (o, m, or p 4-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, or (C₂-C₂₀) alkenylene; R² is independently hydrogen, (C₁-C₁₂) alkyl or (C₆-C₁₀) aryl or a protecting group; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol or bicyclic-fragments of 1,4:3,6-dianhydrohexitols of structural formula (II), and combinations thereof; and R⁷ is independently (C₁-C₂₀) alkyl or (C₂-C₂₀) alkenyl;

or a poly(ester urethane) (PEUR) having a chemical formula described by structural formula (IV), 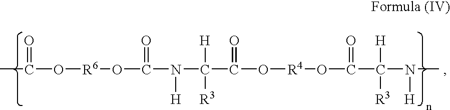
wherein n ranges from about 5 to about 150; wherein the R³s are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; R⁴ is selected from the group consisting of (C₂-C₂₀) alkylene, (C₂-C₂₀) alkenylene or alkyloxy, a residue of a saturated or unsaturated therapeutic diol, bicyclic-fragments of 1,4:3,6-dianhydrohexitols of structural formula (II); and combinations thereof, and R⁶ is independently selected from (C₂-C₂₀) alkylene, (C₂-C₂₀) alkenylene or alkyloxy, bicyclic-fragments of 1,4:3,6-dianhydrohexitols of general formula (II), and combinations thereof;

or a PEUR having a chemical structure described by general structural formula (V) 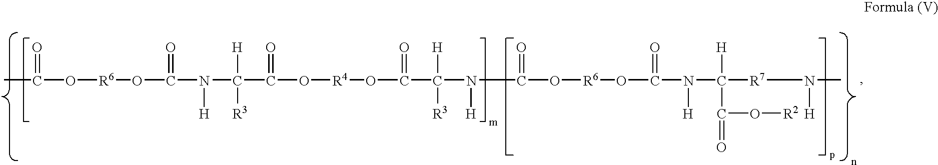
wherein n ranges from about 5 to about 150, m ranges about 0.1 to about 0.9: p ranges from about 0.9 to about 0.1; R² is independently selected from hydrogen, (C₆-C₁₀) aryl (C₁-C₂₀) alkyl, or a protecting group; the R³s in an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is selected from the group consisting of (C₂-C₂₀) alkylene, (C₂-C₂₀) alkenylene or alkyloxy, a residue of a saturated or unsaturated therapeutic diol and bicyclic-fragments of 1,4:3,6-dianhydrohexitols of structural formula (II) and combinations thereof; R⁶ is independently selected from (C₂-C₂₀) alkylene, (C₂-C₂₀) alkenylene or alkyloxy, bicyclic-fragments of 1,4:3;6-dianhydrohexitols of general formula (II), a residue of a saturated or unsaturated therapeutic diol, and combinations thereof; and R⁷ is independently (C₁-C₂₀) alkyl or (C₂-C₂₀) alkenyl;

or a poly(ester urea) (PEU) polymer having a chemical formula described by general structural formula (VI): 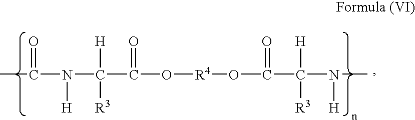
wherein n is about 10 to about 150; the R³s within an individual n monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; a bicyclic-fragment of a 1,4:3,6-dianhydrohexitol of structural formula (II), and combinations thereof;

or a PEU having a chemical formula described by structural formula (VII) 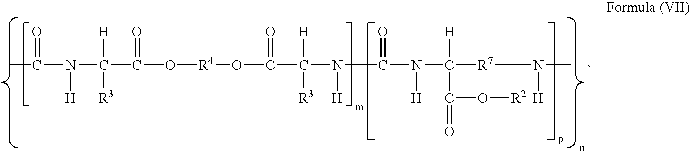
wherein m is about 0.1 to about 1.0; p is about 0.9 to about 0.1; n is about 10 to about 150; R² is independently hydrogen, (C₁-C₁₂) alkyl or (C₆-C₁₀) aryl; the R³s within an individual m monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; a bicyclic-fragment of a 1,4:3,6-dianhydrohexitol of structural formula (II), and combinations thereof; and R⁷ is independently (C₁-C₂₀) alkyl or (C₂-C₂₀) alkenyl.

In another embodiment, the invention provides micelle-forming polymer particle delivery compositions for delivery of a macromolecular biologic dispersed in particles of a biodegradable polymer. In this embodiment the polymer is made of a hydrophobic section containing a biodegradable polymer having a chemical structure described by structural formula (I) or (III-VII) joined to a water soluble section. The water soluble section is made of at least one block of ionizable poly(amino acid), or repeating alternating units of i) polyethylene glycol, polyglycosaminoglycan, or polysaccharide; and ii) at least one ionizable or polar amino acid. The repeating alternating units have substantially similar molecular weights and the molecular weight of the polymer is in the range from about 10 kDa to 300 kDa.

In still another embodiment, the invention provides methods for delivering a substantially structurally intact macromolecular biologic to a subject by administering to the subject in vivo an invention polymer particle delivery composition comprising a liquid dispersion of polymer particles having dispersed therein at least one macromolecular biologic, which particles biodegrade by enzymatic action to release the macromolecular biologic in vivo with substantially native activity over time.

In yet another embodiment, the invention provides methods for delivering polymer particles containing a macromolecular biologic with substantial native activity to a local site in the body of a subject. In this embodiment the invention methods involve delivering a dispersion of particles of a polymer comprising at least one or a blend of those described by structural formulas (I) or (III-VII) herein, wherein the particles have a macromolecular biologic dispersed therein, into an in vivo site in the body of the subject, where the injected particles agglomerate to form a polymer depot of particles of increased size for controlled release of the macromolecular biologic.

### BRIEF DESCRIPTION OF THE FIGURES

FIG. 1 is a schematic drawing illustrating a water soluble covering molecule coating the exterior of a polymer particle.

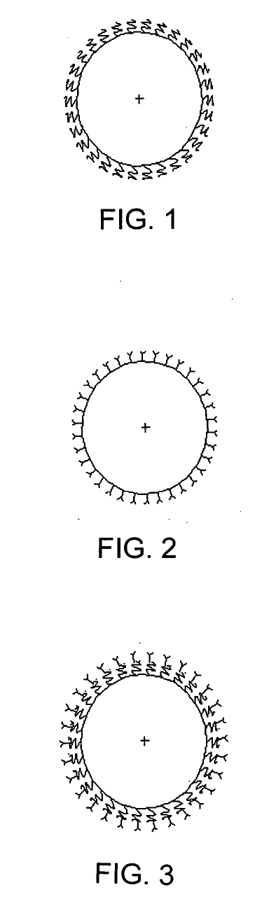

FIG. 2 is a schematic drawing illustrating a bioactive agent coating the exterior of a polymer particle.

FIG. 3 is a schematic drawing illustrating a water-soluble polymer coating applied to the exterior of a polymer particle to which is attaching a bioactive agent.

FIGS. 4-9 are schematic drawings representing invention polymer particles with active agents dispersed therein by double and triple emulsion procedures described herein. FIG. 4 shows a polymer particle encapsulating drug in water formed by double emulsion technique. FIG. 5 shows a polymer particle formed by double emulsion in which drops of water in which drug is dissolved are matrixed within the polymer particle. FIG. 6 shows a polymer particle formed by a triple emulsion technique in which a drug dispersed in water is encapsulated within a polymer coating forming the particle. FIG. 7 shows a polymer particle formed by a triple emulsion technique in which smaller particles of polymer containing dispersed drug are matrixed in water and coated with a polymer coating forming the particle. FIG. 8 shows a polymer particle formed of drug matrixed in the polymer forming the particle. FIG. 9 shows a drug/first polymer mixture encapsulated within a coating of a second polymer in which the mixture is not soluble.

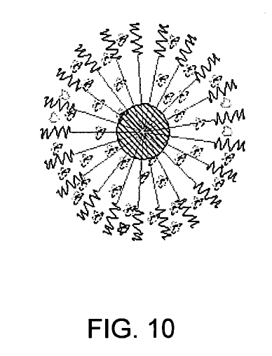

FIG. 10 is a schematic drawing illustrating invention micelles containing dispersed active agents, as described herein.

FIG. 11 is a schematic drawing illustrating micro-crystallites of biologic macromolecular promoters being stabilized by promoter-polymer conjugation. 1=oligomerization (Zn-hexamers for insulin; 2=crystallization of promoters and oligomers; 3=polymer chain network via oligomer; 4=polymer chain network via promoter. White=promoters not conjugated; black=promoters conjugated to polymer chain; circles=zinc.

FIG. 12 is a graph showing a decrease in blood glucose level (FBG) resulting from administration to fasting hypoglycemic mice of biologically active insulin released from polymer particles made according to the invention. No change in FBG is a value of 1.0. Glucose normalized to polymer control.

FIG. 13 is a graph showing a decrease in blood glucose level (FBG) resulting from administration to fasting hypoglycemic rats of biologically active insulin released from polymer particles made according to the invention. No change in FBG is a value of 1.0.

FIGS. 14 A and B show a series of graphs that summarize changes in blood glucose and insulin in normoglycemic rats resulting from subcutaneous injections of free insulin or administration of insulin-polymer conjugate particles into the duodenum. FIG. 14A (1)=portal vein insulin; FIG. 14A (2)=SubQ tail vein insulin; FIG. 14B (3)=20 IU/kg PEA-insulin particles, portal vein insulin; FIG. 14B (4)=20 IU/kg PEA-insulin particles, tail vein insulin

### A DETAILED DESCRIPTION OF THE INVENTION

The invention provides a bio-compatible, biodegradable polymer delivery composition for macromolecular biologics. The polymers used are not hydrophilic overall (i.e. are not water-soluble), and thereby more protectively wrap the biologic than a hydrogel. Yet, unlike truly hydrophobic polymers, these polymers stabilize the three-dimensional structure of cargo biologic macromolecules via the same non-covalent forces that are found within native macromolecular biologics, and aggregates thereof to substantially maintain native activity of the biologic macromolecules. These stabilizing forces arise from discrete hydrophobic segments along the polymer chains, which give rise to short-range dispersion forces, and charged or partially charged regions of the polymer, which give rise to localized ionic interactions, including hydrogen bonds. In particular, in the invention polymer delivery compositions for macromolecular biologics, hydrogen bonding may occur directly between polymer and macromolecular biologic, or may be bridged via a discrete water molecule in a manner equivalent to the slowly exchangeable, bound water molecules found at the surface of native biologic macromolecules and which form a bridge between macromolecules in aggregates thereof, such as crystals.

A “macromolecular biologic” as the term is used herein includes proteins, polypeptides, oligopeptides, nucleic acids polynucleotides and oligonucleotides, macromolecular lipids and polysaccharides, whose bioactivity depends upon a unique three-dimensional (e.g., folded) structure of the molecule. This three-dimensional molecular structure is substantially maintained by specific non-covalent bonding interactions, such as hydrogen bonding and hydrophobic bonding interactions between atoms (hydrophobicity). A “macromolecular biologic” can be either naturally occurring or man-made by any method known in the art.

As used herein, “bioactive agent” means any molecule other than a “macromolecular biologic” that is produced artificially or biologically and that affects a biological process with a therapeutic or palliative result when co-administered. Included without limitation, are short peptides, factors, small molecule drugs, sugars, lipids and whole cells. The macromolecular biologics and, optionally, bioactive agents are administered in polymer particles having a variety of sizes and structures suitable to meet differing therapeutic goals and routes of administration. The “bioactive agent” is not incorporated into the polymer backbone.

As used herein, the terms “amino acid” and “α-amino acid” mean a chemical compound containing an amino group, a carboxyl group and a pendent R group, such as the R³ groups defined herein. As used herein, the term “biological α-amino acid” means the amino acid(s) used in synthesis are selected from phenylalanine, leucine, glycine, alanine, valine, isoleucine, methionine, proline, or a mixture thereof. Lysine and ornithine are also included when R⁷ is hydrogen, albeit incorporated in the polymer backbone adirectionally, i.e., in a direction other than that normally found in a peptide bond.

As used herein, a “therapeutic diol” means any diol molecule, whether synthetically produced, or naturally occurring (e.g., endogenously) that affects a biological process in a mammalian individual, such as a human, in a therapeutic or palliative manner when administered to the mammal.

As used herein, the term “residue of a therapeutic diol” means a portion of a therapeutic diol, as described herein, which portion excludes the two hydroxyl groups of the diol. The corresponding therapeutic diol containing the “residue” thereof is used in synthesis of the polymer compositions. The residue of the therapeutic diol is reconstituted in vivo (or under similar conditions of pH, aqueous media, and the like) to the corresponding diol upon release from the backbone of the polymer by biodegradation in a controlled manner that depends upon the properties of the PEA, PEUR or PEU polymer selected to fabricate the composition, which properties are as known in the art and as described herein.

The term, “biodegradable” as used herein to describe the polymers used in the invention polymer particle delivery compositions, means the polymer is capable of being metabolized into innocuous products, such as amino acids, during the normal functioning of the body. In one embodiment, the entire polymer particle delivery composition is biodegradable. The preferred biodegradable polymers have hydrolyzable and/or enzymatically cleavable ester and enzymatically cleavable amide linkages that provide the biodegradability, and are typically chain terminated predominantly with amino groups. Optionally, these amino termini can be acetylated or otherwise capped by conjugation to any other acid-containing, biocompatible molecule, to include without restriction organic acids, bio-inactive biologics and bio-active compounds such as adjuvant molecules.

The polymer particle delivery compositions can be formulated to provide a variety of properties. In one embodiment, the polymer particles are fabricated to agglomerate, forming a time-release polymer depot for local delivery of dispersed macromolecular biologics to surrounding tissue/cells when injected in vivo, for example subcutaneously, intramuscularly, or into an interior body site, such as an organ. For example, invention polymer particles of sizes capable of passing through pharmaceutical syringe needles ranging in size from about 19 to about 27 Gauge, for example those having an average diameter in the range from about 1 μm to about 200 μm, can be injected into an interior body site, and will agglomerate to form particles of increased size that form the depot to dispense the macromolecular biologic(s) locally. In other embodiments, the biodegradable polymer particles act as a carrier for the macromolecular biologic into the circulation for targeted and timed release systemically. Invention polymer particles in the size range of about 10 nm to about 500 nm will enter directly into the circulation for such purposes.

The biodegradable polymers used in the invention polymer particle delivery composition can be designed to tailor the rate of biodegradation of the polymer to result in continuous delivery of the macromolecular biologic over a selected period of time. For instance, typically, a polymer depot, as described herein, will biodegrade over a period of about twenty-four hours, about seven days, about thirty days, or about ninety days, or longer. Longer time spans are particularly suitable for providing a delivery composition that eliminates the need to repeatedly inject the composition to obtain a suitable therapeutic or palliative response.

The present invention utilizes biodegradable polymer particle-mediated delivery techniques to deliver a wide variety of macromolecular biologics and, optionally, bioactive agents, in treatment of a wide variety of diseases and disease symptoms. Although certain of the individual components of the polymer particle delivery composition and methods described herein were known, it was unexpected and surprising that such combinations would enhance the efficiency of time release delivery of the macromolecular biologics beyond levels achieved when the components were used separately.

The biodegradable polymers useful in forming the invention biocompatible polymer particle delivery compositions include those comprising at least one amino acid conjugated to at least one non-amino acid moiety per repeat unit. In the PEA, PEUR and PEU polymers useful in practicing the invention, multiple different α-amino acids can be employed in a single polymer molecule. The term “non-amino acid moiety” as used herein includes various chemical moieties, but specifically excludes amino acid derivatives and peptidomimetics as described herein. In addition, the polymers containing at least one amino acid are not contemplated to include poly(amino acid) segments, including naturally occurring polypeptides, unless specifically described as such. In one embodiment, the non-amino acid is placed between two adjacent amino acids in the repeat unit. The polymers may comprise at least two different amino acids per repeat unit, for example per n monomer, and a single polymer molecule may contain multiple different α-amino acids in the polymer molecule, depending upon the size of the molecule. In another embodiment, the non-amino acid moiety is hydrophobic. The polymer may also be a block co-polymer. In another embodiment, the polymer is used as one block in di- or tri-block copolymers, which are used to make micelles, as described below.

The PEAs, PEURs and PEUs used in practice of the invention can have built-in functional groups on side chains, and these built-in functional groups can react with other chemicals and lead to the incorporation of additional functional groups to expand the functionality of PEA, PEUR or PEU further. Therefore, such polymers used in the invention methods are ready for reaction with other chemicals having a hydrophilic structure to increase water solubility of the particles and, optionally, with bioactive agents and covering molecules, without the necessity of prior modification.

In addition, the polymers used in the invention polymer particle delivery compositions display minimal hydrolytic degradation when tested in a saline (PBS) medium, but in an enzymatic solution, such as chymotrypsin or CT, a uniform erosive behavior has been observed.

In one embodiment, the invention provides a polymer particle delivery composition in which at least one macromolecular biologic is dispersed in a biodegradable polymer comprising at least one or a blend of the following: a PEA having a chemical structure described by structural formula (I), 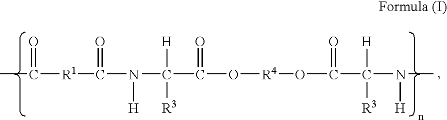
wherein n ranges from about 5 to about 150; R¹ is independently selected from residues of α,ω-bis-(o, m, or p-carboxyphenoxy) (C₁-C₈) alkane, 3,3′-(alkanedioyldioxy) dicinnamic acid or 4,4′-(alkanedioyldioxy) dicinnamic acid, (C₂-C₂₀) alkylene, and (C₂-C₂₀) alkenylene; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; and R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol, bicyclic-fragments of 1,4:3,6-dianhydrohexitols of structural formula (II), and combinations thereof, 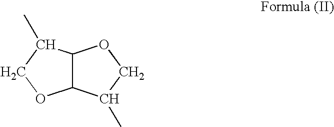
or a PEA polymer having a chemical formula described by structural formula III: 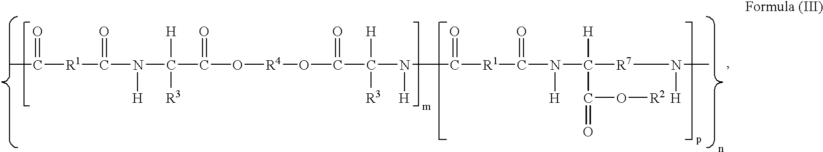
wherein n ranges from about 5 to about 150, m ranges about 0.1 to 0.9: p ranges from about 0.9 to 0.1; wherein R¹ is independently selected from residues of α,ω-bis (o, m, or p-carboxyphenoxy) (C₁-C₈) alkane, 3,3′-(alkanedioyldioxy) dicinnamic acid or 4,4′-(alkanedioyldioxy) dicinnamic acid, (C₂-C₂₀) alkylene, or (C₂-C₂₀) alkenylene; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; R⁴ is independently selected from the group consisting of (C₂-C₂₀) alkylene, (C₂-C₂₀) alkenylene, (C₂-C₈) alkyloxy, (C₂-C₂₀) alkylene, a residue of a saturated or unsaturated therapeutic diol, bicyclic-fragments of 1,4:3,6-dianhydrohexitols of structural formula (II), and combinations thereof; and R⁷ is independently (C₁-C₂₀) alkyl or (C₂-C₂₀) alkenyl.

For example, in one alternative in the PEA polymer used in the invention particle delivery composition, at least one R¹ is a residue of α,ω-bis (o, m, or p-carboxyphenoxy) (C₁-C₈) alkane, 3,3′-(alkanedioyldioxy)dicinnamic acid, or 4,4′-(alkanedioyldioxy)dicinnamic acid and R⁴ is a bicyclic-fragment of a 1,4:3,6-dianhydrohexitol of general formula (II). In another alternative, R¹ in the PEA polymer is either a residue of α,ω-bis (o, m, or p-carboxyphenoxy) (C₁-C₈) alkane, 3,3′-(alkanedioyldioxy)dicinnamic acid, or 4,4′-(alkanedioyldioxy)dicinnamic acid. In yet another alternative, in the PEA polymer R¹ is a residue α,ω-bis (o, m, or p-carboxyphenoxy) (C₁-C₈) alkane, such as 1,3-bis (4-carboxyphenoxy)propane (CPP), 3,3′-(alkanedioyldioxy)dicinnamic acid or 4,4′-(adipoyldioxy)dicinnamic acid and R⁴ is a bicyclic-fragment of a 1,4:3,6-dianhydrohexitol of general formula (II), such as DAS. In yet another alternative in the PEA, R⁷ is independently (C₃-C₆ alkyl, for example, —(CH₂)₄—.

In another embodiment, the polymer comprises a PEUR having a chemical formula described by structural formula (IV), 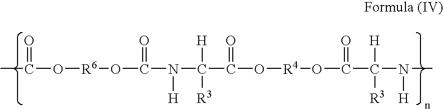
wherein n ranges from about 5 to about 150; wherein R³s in independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is selected from the group consisting of (C₂-C₂₀) alkylene, (C₂-C₂₀) alkenylene or alkyloxy, a residue of a saturated or unsaturated therapeutic diol and bicyclic-fragments of 1,4:3,6-dianhydrohexitols of structural formula (II); and R⁶ is independently selected from (C₂-C₂₀) alkylene, (C₂-C₂₀) alkenylene or alkyloxy, bicyclic-fragments of 1,4:3,6-dianhydrohexitols of general formula (II), an effective amount of a residue of a saturated or unsaturated therapeutic diol, and combinations thereof,

or a PEUR having a chemical structure described by general structural formula (V) 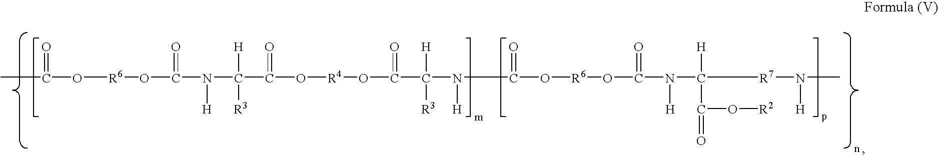
wherein n ranges from about 5 to about 150, m ranges about 0.1 to about 0.9: p ranges from about 0.9 to about 0.1; R² is independently selected from hydrogen, (C₆-C₁₀) aryl (C₁-C₂₀) alkyl, or a protecting group; the R³s in an individual m monomer are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; R⁴ is selected from the group consisting of (C₂-C₂₀) alkylene, (C₂-C₂₀) alkenylene or alkyloxy, and bicyclic-fragments of 1,4:3,6-dianhydrohexitols of structural formula (II); R⁶ is independently selected from (C₂-C₂₀) alkylene, (C₂-C₂₀) alkenylene or alkyloxy, bicyclic-fragments of 1,4:3,6-dianhydrohexitols of general formula (II), and combinations thereof; and R⁷ is independently (C₁-C₂₀) alkyl or (C₂-C₂₀) alkenyl.

In one alternative in the PEUR polymer, at least one of R⁴ is a bicyclic fragment of 1,4:3,6-dianhydrohexitol (formula (II)), such as 1,4:3,6-dianhydrosorbitol (DAS); or R⁶ is a bicyclic fragment of 1,4:3,6-dianhydrohexitol, such as 1,4:3,6-dianhydrosorbitol (DAS). In still alternative in the PEUR polymer, R⁴ and/or R⁶ is a bicyclic fragment of 1,4:3,6-dianhydrohexitol, such as 1,4:3,6-dianhydrosorbitol (DAS). In yet another alternative in the PEUR, R⁷ is independently (C₃-C₆ alkyl, for example, —(CH₂)₄—.

In yet another embodiment the polymer in the invention particle delivery composition comprises a PEU having a chemical formula described by general structural formula (VI): 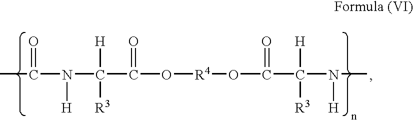
wherein n is about 10 to about 150; the R³s within an individual n monomer are independently selected from hydrogen, (C₁-C₆) alkyl, (C₂-C₆) alkenyl, (C₂-C₆) alkynyl, (C₆-C₁₀) aryl (C₁-C₂₀) alkyl and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), and combinations thereof;

or a PEU having a chemical formula described by structural formula (VII) 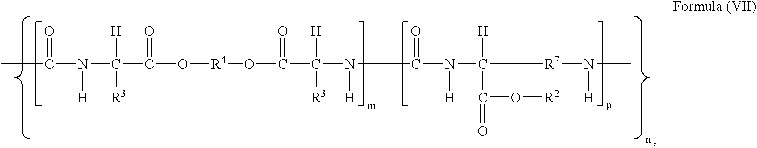
wherein m is about 0.1 to about 1.0; p is about 0.9 to about 0.1; n is about 10 to about 150; R² is independently hydrogen, (C₁-C₁₂) alkyl or (C₆-C₁₀) aryl or other protective group; and the R³s within an individual m monomer are independently selected from hydrogen, (C₁-C₆) alkyl, (C₂- C₆) alkenyl, (C₂-C₆) alkynyl, (C₆-C₁₀) aryl (C₁-C₂₀)alkyl, —(CH₂)₃— and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, an effective amount of a residue of a saturated or unsaturated therapeutic diol; or a bicyclic-fragment of a 1,4:3,6-dianhydrohexitol of structural formula (II); and R⁷ is independently (C₁-C₂₀) alkyl or (C₂-C₂₀) alkenyl. In yet another alternative in the PEA, R⁷ is independently (C₃-C₆) alkyl, for example, —(CH₂)₄—.

Suitable protecting groups for use in practice of the invention include t-butyl and others as are known in the art. Suitable bicyclic-fragments of 1,4:3,6-dianhydrohexitols can be derived from sugar alcohols, such as D-glucitol, D-mannitol, and L-iditol. For example, 1,4:3,6-dianhydrosorbitol (isosorbide, DAS) is particularly suited for use as a bicyclic-fragment of 1,4:3,6-dianhydrohexitol.

These PEU polymers can be fabricated as high molecular weight polymers useful for making the invention polymer particle delivery compositions for delivery to humans and other mammals of a variety of pharmaceutical and biologically active agents. The invention PEUs incorporate hydrolytically cleavable ester groups and non-toxic, naturally occurring monomers that contain α-amino acids in the polymer chains. The ultimate biodegradation products of PEUs will be α-amino acids (whether biological or not), diols, and CO₂. In contrast to the PEAs and PEURs, the invention PEUs are crystalline or semi-crystalline and possess advantageous mechanical, chemical and biodegradation properties that allow formulation of completely synthetic, and hence easy to produce, crystalline and semi-crystalline polymer particles, for example nanoparticles.

For example, the PEU polymers used in the invention polymer particle delivery compositions have high mechanical strength, and surface erosion of the PEU polymers can be catalyzed by enzymes present in physiological conditions, such as hydrolases.

In one alternative in the PEU polymer, at least one R¹ is a bicyclic fragment of a 1,4:3,6-dianhydrohexitol, such as 1,4:3,6-dianhydrosorbitol (DAS).

Suitable protecting groups for use in practice of the invention include 1-butyl and others as are known in the art. Suitable bicyclic-fragments of 1,4:3,6-dianhydrohexitols can be derived from sugar alcohols, such as D-glucitol, D-mannitol, and L-iditol. For example, dianhydrosorbitol is particularly suited for use as a bicyclic-fragment of 1,4:3,6-dianhydrohexitol.

In one alternative, the R³s in at least one n monomer are CH₂Ph and the α-amino acid used in synthesis is L-phenylalanine. In alternatives wherein the R³s within a monomer are —CH₂—CH(CH₃)₂, the polymer contains the α-amino acid, leucine. By varying the R³s, other α-amino acids can also be used, e.g., glycine (when the R³s are —H), proline (when the R³s are ethylene amide); alanine (when the R³s are —CH₃), valine (when the R³s are —CH(CH₃)₂), isoleucine (when the R³s are —CH(CH₃—CH₂—CH₃), phenylalanine (when the R³s are —CH₂—C₆H₅); lysine (when the R³s are —(CH₂)₄—NH₂); or methionine (when the R³s are —(CH₂)₂SCH₃).

In yet a further embodiment wherein the polymer is a PEA, PEUR or PEU of formula I or III-VII, at least one of the R³s further can be —(CH₂)₃— and the R³s cyclize to form the chemical structure described by structural formula XV:

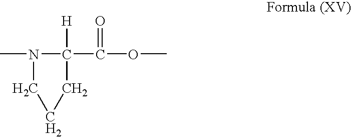

When the R³s are —(CH₂)₃, an α-imino acid analogous to pyrrolidine-2-carboxylic acid (proline) is used.

The PEAs, PEURs and PEUs are biodegradable polymers that biodegrade substantially by enzymatic action so as to release the dispersed macromolecular biologics over time. Due to structural properties of the polymer used, the invention polymer particle delivery compositions provide for stable loading of macromolecular biologics while preserving the three dimensional structure thereof and, hence, the bioactivity.

Polymers suitable for use in the practice of the invention bear functionalities that allow optional covalent attachment of bioactive agent(s) or covering molecule(s) to the polymer. For example, a polymer bearing carboxyl groups can readily react with an amino moiety of a peptide, thereby covalently bonding a peptide to the polymer via the resulting amide group. As will be described herein, the biodegradable polymer and, optionally, any bioactive agent, may contain numerous complementary functional groups that can be used to covalently attach the optional bioactive agent to the biodegradable polymer.

The polymer in the invention polymer particle delivery composition plays an active role in the treatment processes at the site of local injection by holding the macromolecular biologic and any bioactive agent at the site of injection for a period of time sufficient to allow the individual's endogenous processes to interact with the macromolecular biologic and any bioactive agent present, while slowly releasing the particles or polymer molecules containing such macromolecular biologics and optional agents during biodegradation of the polymer. The fragile macromolecular biologic is protected by the slowly biodegrading polymer to increase the half-life and persistence of the macromolecular biologic(s).

In addition, the polymers disclosed herein (e.g., those having structural formulas (I and III-VII), upon enzymatic degradation, provide biological or non biological amino acids, while the other breakdown products can be metabolized in biochemical pathways equivalent to those for fatty acids and sugars. Uptake of the polymer particles in vivo with macromolecular biologic is safe: studies have shown that the subject can metabolize/clear the polymer degradation products. These polymers and the invention polymer particle delivery compositions are, therefore, substantially non-inflammatory to the subject both at the site of injection, apart from the trauma caused by injection itself, and systemically, and are particularly suited for oral or intra-nasal delivery.

Enhancement of Biologic Loading and Stability by Aggregation, Oligomerization or Crystallization

Due to the hydrocarbon segments contained therein, the synthetic PEAs, PEURs, and PEUs described herein are not soluble in water. However, they are partially wettable, probably because individual water molecules can hydrogen-bond to the amino acid residues, and thereby form hydrogen bonded bridges to more water molecules. It is believed that these bound water molecules are important for the stabilization of interactions between the polymer and macromolecular biologics, in much the same way as discrete, bound water molecules have been demonstrated to be essential for the stabilization of macromolecular biologic structures and of higher order structures, such as oligomers and crystals.

Crystalline arrays of biological molecules in which the crystallites are formed under mild conditions represent natural or quasi-natural configurations that can achieve optimal packing density, while stabilizing the macromolecular structure. Indeed, some proteins, e.g. pro-insulin, are naturally preserved within storage granules as micro-crystalline aggregates.

In nature, many macromolecular biologics exist as a quaternary structure, which structure often represents the active biological configuration. Examples of macromolecular biologics that exist as a quaternary structure include some nucleic acids (anti-parallel, double helical dimers), many gene-regulatory proteins (DNA-binding dimers of two promoters), the transport proteins hemoglobin and transthyretin (each a quartet of promoters), the enzyme aspartate transcarbamoylase (six regulatory plus catalytic promoters), iscosahedral virus coats (multiples of sixty promoters), helical virus coats (Tobacco Mosaic virus has 2130 promoters), and cell-structural assemblies, such as actin and tubulin cables (composed of many thousands of promoters).

Two or more such identical protein molecules or promoters bind together non-covalently, but specifically, so as to form a protein oligomer. The spatial arrangement of the promoters is called the quaternary structure of the oligomer. In most biological oligomers, the promoters are spatially related by simple rotational symmetries. However, many oligomeric proteins crystallize with more than one promoter in the crystallographic asymmetric unit, so these symmetries are not necessarily exact. An example of a quaternary configuration of promoters commonly observed in crystal structures of oligomeric proteins is that of dimers that are related by additional rotational symmetries. The resulting oligomer, which may, or may not represent the biologically active configuration, is more stable and has a lower free-energy minimum than a simple translational crystalline aggregate of the promoter. For example, human insulin readily dimerizes and, in the presence of zinc atoms, three dimers assemble around a three-fold axis of symmetry to form a stable hexamer of molecules. Under suitable conditions, these soluble hexamers can be aggregated to form crystals in which hexamer-hexamer interactions are further stabilized by zinc atoms. For macromolecular biologics other than insulin, atoms of other transition metals or calcium may facilitation aggregation of oligomers to form crystals.

The example of crystallization of insulin is described herein to illustrate an important general feature of crystallization of macromolecular biologics, such as proteins. The non-covalent electronic forces that bind the crystal are similar in type and strength to those that stabilize the quaternary structure of an oligomer, and that indeed maintain the three-dimensional folding of the protein molecule (i.e., the promoter) itself.

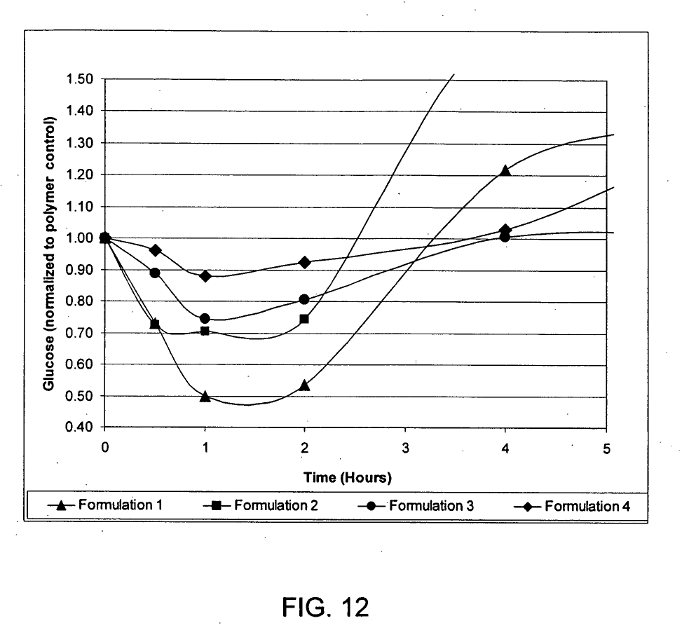

Thus, the three-dimensional folded structure of a macromolecular biologic can be preserved in the invention PEA, PEUR and PEU polymer particle delivery compositions by a combination of hydrophobic and ionic bonding of the macromolecular biologic: 1) to the polymer, 2) to spatially neighboring copies of the macromolecular biologic itself (i.e., micro-crystallization, with or without oligomerization), and, optionally, 3) to spatially neighboring copies of the macromolecular biologic itself (i.e., crystallization, with or without oligomerization) in which, a minority of promoters have been conjugated to the polymer. Multivalent biologically active molecules (i.e. macromolecular biologics with more than one site for conjugation, as in Example 10 herein) within molecular weight range from about 100 to about 1,000,000 Da, can partially crosslink the polymer and provide additional stabilization of the system. As illustrated in FIG. 11 and exemplified in the Examples herein, it is envisioned that these polymer-conjugated promoters act as seed molecules, promoting the crystallization, with or without oligomerization and under mild conditions, of surrounding free promoters, thereby stabilizing the three-dimensional structure of the promoters, and so preserving native biological activity of the macromolecular biologic(s).

Not all macromolecular biologics will form crystals or oligomers in this way, but many will form aggregates that maintain native activity of the molecules. For example, oligonucleotides form two-molecule aggregates through normal base pairing in the sense and antisense strands.

Accordingly in one embodiment the invention provides polymer particle delivery compositions in which at least one macromolecular biologic is conjugated to a biodegradable polymer via active groups therein, such as the PEAs, PEURs or PEUs having a chemical formula described by any one of structural formulas (I) or (III-VII). Conjugation of the macromolecular biologic to the polymer is illustrated herein in the Examples by conjugation of insulin or ovalbumin to PEA using such conjugation chemistry as the DMSO protein/polymer solvated activated ester method. Alternatively, the solvent HFIP-activated ester method can be used to create the polymer-biologic conjugate using the protein ovalbumin. The macromolecular biologic-containing conjugate can then be incorporated into an aggregate or oligomer (e.g., an insulin hexamer with zinc) and crystallized using a dialysis method as described in the Examples herein, and as known in the art.

To protect the three dimensional structure of the macromolecular biologic in the conjugate, the conjugate can be coated with or matrixed within a coating polymer, such as a PEA of structure I or III or a PEUR of structure IV or V, or a PEU of structure VI or VII. Solution lyophilization is used to coat or matrix the conjugate using such solvents as Dioxane, Dioxane/HFIP or HFIP, as illustrated herein by Examples 10 and 11.

Moreover, the three-dimensional structure of the active macromolecular biologic in the conjugate can be protected by encapsulation of the conjugate within a PEA, PEUR or PEU polymer particle using a water in organic solvent (w/o emulsion) method. Alternatively, an immiscible solvent technique employing an organic oil and a polar organic solvent (o/o emulsion) method can be used to form particles, such as nanoparticles, that encapsulate the macromolecular biologic, as a promoter, an oligomer, or as a crystal of oligomers (as illustrated in FIG. 11). The single, double and triple emulsion techniques described below are all applicable for this purpose.

In another embodiment, invention polymer particle delivery compositions that are intended for oral delivery of insulin optionally may further comprise at least one bile salt, an endogenous permeation enhancer, dispersed in the amino acid based PEA or PEUR polymer(s) of the microparticles described herein. In this embodiment, PEA and PEUR microparticles can be used to orally deliver insulin because they are expected to deliver concentrated amounts of insulin to the microvilli of the intestine for absorption by protecting it from proteolysis. The concentrated amounts of insulin in the invention compositions result from formation of a crystalline form of insulin-hexamers bound on insulin conjugated to the polymer, as described herein. Under normal physiological conditions in the intestine, absorption of insulin by the columnar epithelium is very low. In this alternative embodiment of the invention, bile salts matrixed in the polymer that sequesters the insulin-hexamers, enhances permeability of insulin across the intestinal wall and this is most likely due to the presence of sterol-like molecules at the surface of the microparticles. Thus, the polymer in the invention polymer particle delivery composition contributes stability to and protects insulin within the polymer-bile salt-insulin microspheres as it travels through the lumen of the intestine, while the bile salts enhance rapid release of insulin from the microparticles when subjected to the physiological conditions of the brush border of the intestine.

In fact, it is expected that the released insulin will be protected by spontaneous formation of micelles around the insulin and this is hypothesized to be the correct mechanism based on the physiology of bile salts in the gut forming micelles, which aid the delivery of insulin through the mucosal cells of the villi. Whatever the exact mechanism, a concentrated bolus of insulin can be quickly released by the microspheres into the mucous and glycocalyx layers coating the simple columnar epithelium. From there, the bile salt-coated insulin should efficiently diffuse through the epithelial cells and lamina propria as chylomicron-like particles and be rapidly transported by blood flow through the hepatic portal vein to the hepatocytes of the liver, so as to reduce the blood levels of postprandial glucose.

In the embodiment of the invention in which one or more bile salts are matrixed in the PEA or PEUR microparticle that sequesters the insulin, advantage is taken of a major circulatory pathway, the enterohepatic circulatory pathway, for insulin uptake from the small and large intestine to the liver. This pathway is important in recycling bile salts through the gut to aid in the digestion and absorption of food. The transport of intact biologically active macromolecules from the intestinal lumen into the blood circulation is a unique phenomenon which differs from the regular process of food digestion and absorption. Intestinal absorption of bioactive peptides and various proteins has been reported (Ziv, E., et al. Biochemical Pharmacology (1987) 36(7):1035-1039). It has been shown that protection against proteolysis is the first step involved in keeping polypeptides and proteins intact in the “hostile” intestinal lumen (See references in Ziv, supra). The second step entails alteration of the mechanisms responsible for selective absorption of small molecules to enable absorption of high molecular weight molecules. Since they are endogenous, these natural and specialized “amphipathic” permeation enhancers are less likely to produce severe side effects in the individual than are other types of amphipathic molecules.

Bile is a hepatic secretion that appears to have two principal functions: first, to promote the digestion and absorption of lipid from the intestine, and second, to enhance elimination of many endogenous and exogenous substances from the blood and liver that are not excreted through the kidneysⁱⁱ. Bile salts, a major constituent of bile, have a concentration in bile between 2 and 45 mM and are acidic sterols, which in mammals are based on the C₂₄ compound, cholic acid. The bile salts useful in the invention include the commonly occurring bile salts based on cholic acid: cholate, chenodeoxycholate and lithocholate, which differ in the number of hydroxyl groups on the cholic acid ring structure. The natural bile salts optionally used in the invention compositions will be reused by the liver for its own production of bile. Re-absorption of such salts occurs mainly in the duodenum and terminal ileum and, after passage across the cells of the small intestinal wall, bile salts return to the liver via the portal circulation. In humans 99% of the bile salt pool is maintained within the enterohepatic circulation and during each 24-h period approximately 40 g (100 mmol) of bile salt is removed from the portal blood by the liver. Excess bile salts are eliminated through the bowel. (Strange, R. C., Physiological Reviews, (1984) 64(4):1055-1102).

Since bile salts reach the liver predominantly via the portal vein, it can be expected that addition of bile salts to the invention composition will significantly contribute to the delivery of the insulin contained therein to hepatocytes, which are arranged in sheets one cell thick and are situated between the afferent and efferent blood supplies. The composition will first contact the sinusoidal surface of the liver cells, which is the site of receptor systems for several hormones, including insulin, glucagon, and bile salts. In fact, for insulin, the sinusoidal surface of liver cells is the primary target in the body. Microvilli on the sinusoidal surface considerably increase the surface area available for an exchange of molecules between blood and liver cells.

Therefore, while insulin in the invention composition is delivered to the sinusoidal side of the hepatocytes to affect the uptake of blood glucose, the bile salts are recycled through the hepatocytes into the bile, and the polymer is biodegraded by enzymes in the gut and perhaps in the circulatory system, making the bile salt-containing embodiment of the invention compositions safe for oral delivery of insulin.

In yet another embodiment, the invention provides micelle-forming polymer particle delivery compositions for delivery of a macromolecular biologic dispersed in particles of a biodegradable polymer. In this embodiment the polymer is made of a hydrophobic section containing a biodegradable polymer having a chemical structure described by structural formula (I) joined to a water soluble section. The water soluble section is made of at least one block of ionizable poly(amino acid), or repeating alternating units of i) polyethylene glycol, polyglycosaminoglycan, or polysaccharide; and ii) at least one ionizable or polar amino acid. The repeating alternating units have substantially similar molecular weights and the molecular weight of the polymer is in the range from about 10 kD to 300 kD.

In still another embodiment, the invention provides methods for delivering a structurally intact macromolecular biologic to a subject by administering to the subject in vivo an invention polymer particle delivery composition in the form of a liquid dispersion of polymer particles comprising a polymer of structural formulas (I), or (III-VII) and having dispersed therein an effective amount of at least one macromolecular biologic, which particles biodegrade by enzymatic action to release the structurally intact macromolecular biologic in vivo over time.

In yet another embodiment, the invention provides methods for delivering polymer particles containing a structurally intact macromolecular biologic to a local site in the body of a subject. In this embodiment the invention methods involve delivering a dispersion of particles of a polymer selected from those described by structural formulas (I), (III), (IV) or (V) herein, wherein the particles have a macromolecular biologic dispersed therein to an in vivo site in the body of the subject, where the injected particles agglomerate to form a polymer depot of particles of increased size for controlled release of the macromolecular biologic.

The term “aryl” is used with reference to structural formulas herein to denote a phenyl radical or an ortho-fused bicyclic carbocyclic radical having about nine to ten ring atoms in which at least one ring is aromatic. In certain embodiments, one or more of the ring atoms can be substituted with one or more of nitro, cyano, halo, trifluoromethyl, or trifluoromethoxy. Examples of aryl include, but are not limited to, phenyl, naphthyl, and nitrophenyl.

The term “alkenylene” is used with reference to structural formulae herein to mean a divalent branched or unbranched hydrocarbon chain containing at least one unsaturated bond in the main chain or in a side chain.

The molecular weights and polydispersities of PEA and PEUR polymers herein are determined by gel permeation chromatography (GPC) using polystyrene standards. More particularly, number and weight average molecular weights (Mₙ and M_{w}) are determined, for example, using a Model 510 gel permeation chromatography (Water Associates, Inc., Milford, Mass.) equipped with a high-pressure liquid chromatographic pump, a Waters 486 UV detector and a Waters 2410 differential refractive index detector. Tetrahydrofuran (THF) is used as the eluent (1.0 mL/min). The polystyrene standards have a narrow molecular weight distribution.

Methods for making polymers of structural formulas containing an α-amino acid in the general formula are well known in the art. For example, for the embodiment of the polymer of structural formula (I) wherein R⁴ is incorporated into an α-amino acid, for polymer synthesis the α-amino acid with pendant R³ can be converted through esterification into a bis-α,ω-diamine, for example, by condensing the α-amino acid containing pendant R³ with a diol HO—R⁴—OH. As a result, di-ester monomers with reactive α,ω-amino groups are formed. Then, the bis-α,ω-diamine is entered into a polycondensation reaction with a di-acid such as sebacic acid, or its bis-activated esters, or bis-acyl chlorides, to obtain the final polymer having both ester and amide bonds (PEA). Alternatively, for example, for polymers of structure (I), instead of the di-acid, an activated di-acid derivative, e.g., bis-para-nitrophenyl diester, can be used as an activated di-acid. Additionally, a bis-di-carbonate, such as bis (p-nitrophenyl) dicarbonate, can be used as the activated species to obtain polymers containing a residue of a di-acid. In the case of PEUR polymers, a final polymer is obtained having both ester and urethane bonds.

More particularly, synthesis of the unsaturated poly(ester-amide)s (UPEAs) useful as biodegradable polymers of the structural formula (I) as disclosed above will be described,

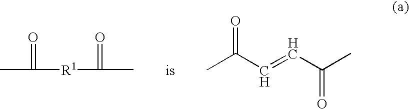

wherein and/or (b) R⁴ is —CH₂—CH═CH—CH₂—. In cases where (a) is present and (b) is not present, R⁴ in (I) is —C₄H₈— or —C₆H₁₂—. In cases where (a) is not present and (b) is present, R¹ in (I) is —C₄H₈— or —C₈H₁₆—.

The UPEAs can be prepared by solution polycondensation of either (1) di-p-toluene sulfonic acid salt of bis (α-amino acid) di-ester of unsaturated diol and di-p-nitrophenyl ester of saturated dicarboxylic acid or (2) di-p-toluene sulfonic acid salt of bis (α-amino acid) diester of saturated diol and di-nitrophenyl ester of unsaturated dicarboxylic acid or (3) di-p-toluene sulfonic acid salt of bis (α-amino acid) diester of unsaturated diol and di-nitrophenyl ester of unsaturated dicarboxylic acid.

Salts of p-toluene sulfonic acid are known for use in synthesizing polymers containing amino acid residues. The aryl sulfonic acid salts are used instead of the free base because the aryl sulfonic salts of bis (α-amino acid) diesters are easily purified through recrystallization and render the amino groups as unreactive ammonium tosylates throughout workup. In the polycondensation reaction, the nucleophilic amino group is readily revealed through the addition of an organic base, such as triethylamine, so the polymer product is obtained in high yield.

For polymers of structural formula (I), for example, the di-p-nitrophenyl esters of unsaturated dicarboxylic acid can be synthesized from p-nitrophenyl and unsaturated dicarboxylic acid chloride, e.g., by dissolving triethylamine and p-nitrophenol in acetone and adding unsaturated dicarboxylic acid chloride dropwise with stirring at −78° C. and pouring into water to precipitate product. Suitable acid chlorides included fumaric, maleic, mesaconic, citraconic, glutaconic, itaconic, ethenyl-butane dioic and 2-propenyl-butanedioic acid chlorides. For polymers of structure (IV) and (V), bis-p-nitrophenyl dicarbonates of saturated or unsaturated diols are used as the activated monomer. Dicarbonate monomers of general structure (XII) are employed for polymers of structural formula (IV), 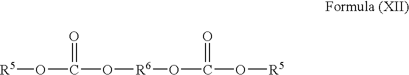
wherein R⁵ is independently (C₆-C₁₀)aryl optionally substituted with one or more nitro, cyano, halo, trifluoromethyl, or trifluoromethoxy; and R⁶ is independently (C₂-C₂₀)alkylene or (C₂-C₂₀) alkyloxy, or (C₂-C₂₀)alkenylene.

The di-aryl sulfonic acid salts of diesters of α-amino acid and unsaturated diol can be prepared by admixing α-amino acid, e.g., p-aryl sulfonic acid monohydrate and saturated or unsaturated diol in toluene, heating to reflux temperature, until water evolution is minimal, then cooling. The unsaturated diols include, for example, 2-butene-1,3-diol and 1,18-octadec-9-en-diol.

Saturated di-p-nitrophenyl esters of dicarboxylic acid and saturated di-p-toluene sulfonic acid salts of bis-α-amino acid esters can be prepared as described in U.S. Pat. No. 6,503,538 B1.

Synthesis of the unsaturated poly(ester-amide)s (UPEAs) useful as biodegradable polymers of the structural formula (I) as disclosed above will now be described. UPEAs having the structural formula (I) can be made in similar fashion to the compound (VII) of U.S. Pat. No. 6,503,538 B I, except that R⁴ of (III) of U.S. Pat. No. 6,503,538 and/or R¹ of (V) of U.S. Pat. No. 6,503,538 is (C₂-C₂₀) alkenylene as described above. The reaction is carried out, for example, by adding dry triethylamine to a mixture of said (III) and (IV) of U.S. Pat. No. 6,503,538 and said (V) of U.S. Pat. No. 6,503,538 in dry N,N-dimethylacetamide, at room temperature, then increasing the temperature to 80° C. and stirring for 16 hours, then cooling the reaction solution to room temperature, diluting with ethanol, pouring into water, separating polymer, washing separated polymer with water, drying to about 30° C. under reduced pressure and then purifying up to negative test on p-nitrophenol and p-toluene sulfonate. A preferred reactant (IV) of U.S. Pat. No. 6,503,538 is p-toluene sulfonic acid salt of Lysine benzyl ester, the benzyl ester protecting group is preferably removed from (II) to confer biodegradability, but it should not be removed by hydrogenolysis as in Example 22 of U.S. Pat. No. 6,503,538 because hydrogenolysis would saturate the desired double bonds; rather the benzyl ester group should be converted to an acid group by a method that would preserve unsaturation. Alternatively, the lysine reactant (IV) of U.S. Pat. No. 6,503,538 can be protected by a protecting group different from benzyl that can be readily removed in the finished product while preserving unsaturation, e.g., the lysine reactant can be protected with t-butyl (i.e., the reactant can be t-butyl ester of lysine) and the t-butyl can be converted to H while preserving unsaturation by treatment of the product (II) with acid.

A working example of the compound having structural formula (I) is provided by substituting p-toluene sulfonic acid salt of bis (L-phenylalanine) 2-butene-1,4-diester for (III) in Example 1 of U.S. Pat. No. 6,503,538 or by substituting di-p-nitrophenyl fumarate for (V) in Example 1 of 6,503,538 or by substituting the p-toluene sulfonic acid salt of bis (L-phenylalanine) 2-butene-1,4-diester for III in Example 1 of U.S. Pat. No. 6,503,538 and also substituting bis-p-nitrophenyl fumarate for (V) in Example 1 of U.S. Pat. No. 6,503,538.

In unsaturated compounds having either structural formula (I) or (IV), the following hold. An amino substituted aminoxyl (N-oxide) radical bearing group, e.g., 4-amino TEMPO, can be attached using carbonyldiimidazol, or suitable carbodiimide, as a condensing agent. Bioactive agents, as described herein, can be attached via the double bond functionality. Hydrophilicity can be imparted by bonding to poly(ethylene glycol) diacrylate.

In yet another aspect, PEA and PEUR polymers contemplated for use in forming the invention polymer particle delivery systems include those set forth in U.S. Pat. Nos. 5,516, 881; 6,476,204; 6,503,538; and in U.S. application Ser. Nos. 10/096,435; 10/101,408; 10/143,572; and 10/194,965; the entire contents of each of which is incorporated herein by reference.

The biodegradable PEA, PEUR and PEU polymers can contain from one to multiple different α-amino acids per polymer molecule and preferably have weight average molecular weights ranging from 10,000 to 125,000; these polymers and copolymers typically have intrinsic viscosities at 25° C., determined by standard viscosimetric methods, ranging from 0.3 to 4.0, for example, ranging from 0.5 to 3.5.

PEA and PEUR polymers contemplated for use in the practice of the invention can be synthesized by a variety of methods well known in the art. For example, tributyltin (IV) catalysts are commonly used to form polyesters such as poly(ε-caprolactone), poly(glycolide), poly(lactide), and the like. However, it is understood that a wide variety of catalysts can be used to form polymers suitable for use in the practice of the invention.

Such poly(caprolactones) contemplated for use have an exemplary structural formula (X) as follows:

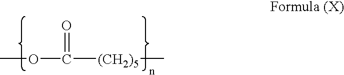

Poly(glycolides) contemplated for use have an exemplary structural formula (XI) as follows:

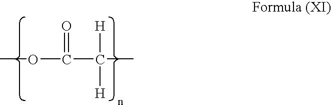

Poly(lactides) contemplated for use have an exemplary structural formula (XII) as follows:

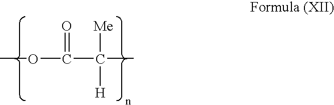

An exemplary synthesis of a suitable poly(lactide-co-ε-caprolactone) including an aminoxyl moiety is set forth as follows. The first step involves the copolymerization of lactide and ε-caprolactone in the presence of benzyl alcohol using stannous octoate as the catalyst to form a polymer of structural formula (XIII).

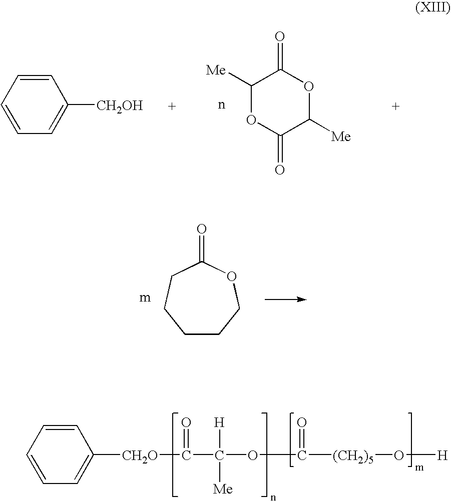

The hydroxy terminated polymer chains can then be capped with maleic anhydride to form polymer chains having structural formula (XIV):

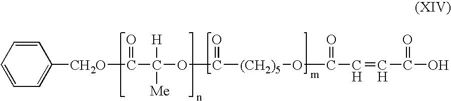

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

In unsaturated compounds having structural formula (VII) for PEU the following hold: An amino substituted aminoxyl (N-oxide) radical bearing group e.g., 4-amino TEMPO, can be attached using carbonyldiimidazole, or suitable carbodiimide, as a condensing agent. Bioactive agents, and the like, as described herein, optionally can be attached via the double bond functionality.

For example, the invention high molecular weight semi-crystalline PEUs having structural formula (I) can be prepared inter-facially by using phosgene as a bis-electrophilic monomer in a chloroform/water system, as shown in the reaction Scheme I below:

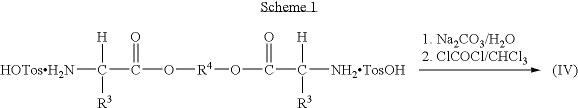

Synthesis of copoly(ester ureas) (PEUs) containing L-Lysine esters and having structural formula (VII) can be carried out by a similar Scheme 2:

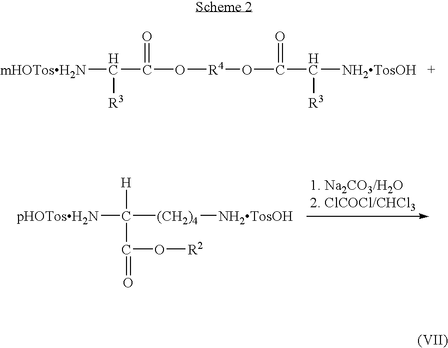

A 20% solution of phosgene (ClCOCl) (highly toxic) in toluene, for example (commercially available (Fluka Chemie, GMBH, Buchs, Switzerland), can be substituted either by diphosgene (trichloromethylchloroformate) or triphosgene (bis (trichloromethyl)carbonate). Less toxic carbonyldiimidazole can be also used as a bis-electrophilic monomer instead of phosgene, di-phosgene, or tri-phosgene.

General Procedure for Synthesis of PEUs

It is necessary to use cooled solutions of monomers to obtain PEUs of high molecular weight. For example, to a suspension of di-p-toluenesulfonic acid salt of bis (α-amino acid)-α,ω-alkylene diester in 150 mL of water, anhydrous sodium carbonate is added, stirred at room temperature for about 30 minutes and cooled to about 2-0° C., forming a first solution. In parallel, a second solution of phosgene in chloroform is cooled to about 15-10° C. The first solution is placed into a reactor for interfacial polycondensation and the second solution is quickly added at once and stirred briskly for about 15 min. Then the chloroform layer can be separated, dried over anhydrous sodium sulfate, and filtered. The obtained solution can be stored for further use.

All the exemplary PEU polymers fabricated were obtained as solutions in chloroform and these solutions are stable during storage. However, some polymers, for example, 1-Phe-4, become insoluble in chloroform after separation. To overcome this problem, polymers can be separated from chloroform solution by casting onto a smooth hydrophobic surface and allowing chloroform to evaporate to dryness. No further purification of obtained PEUs is needed. The yield and characteristics of exemplary PEUs obtained by this procedure are summarized in Table 1 herein.

General Procedure for Preparation of Porous PEUs.

Methods for making the PEU polymers containing α-amino acids in the general formula will now be described. For example, for the embodiment of the polymer of formula (I) or (II), the α-amino acid can be converted into a bis (α-amino acid)-α,ω-diol-diester monomer, for example, by condensing the α-amino acid with a diol HO—R¹—OH. As a result, ester bonds are formed. Then, acid chloride of carbonic acid (phosgene, diphosgene, triphosgene) is entered into a polycondensation reaction with a di-p-toluenesulfonic acid salt of a bis (α-amino acid)-alkylene diester to obtain the final polymer having both ester and urea bonds.

The unsaturated PEUs can be prepared by interfacial solution condensation of di-p-toluenesulfonate salts of bis (α-amino acid)-alkylene diesters, comprising at least one double bond in R¹. Unsaturated diols useful for this purpose include, for example, 2-butene-1,4-diol and 1,18-octadec-9-en-diol. Unsaturated monomer can be dissolved prior to the reaction in alkaline water solution, e.g. sodium hydroxide solution. The water solution can then be agitated intensely, under external cooling, with an organic solvent layer, for example chloroform, which contains an equimolar amount of monomeric, dimeric or trimeric phosgene. An exothermic reaction proceeds rapidly, and yields a polymer that (in most cases) remains dissolved in the transition metals, plus calcium mg, organic solvent. The organic layer can be washed several times with water, dried with anhydrous sodium sulfate, filtered, and evaporated. Unsaturated PEUs with a yield of about 75%-85% can be dried in vacuum, for example at about 45° C.

To obtain a porous, strong material, L-Leu based PEUs, such as 1-L-Leu-4 and 1-L-Leu-6, can be fabricated using the general procedure described below. Such procedure is less successful in formation of a porous bone-like material when applied to L-Phe based PEUs.

The reaction solution or emulsion (about 100 mL) of PEU in chloroform, as obtained just after interfacial polycondensation, is added dropwise with stirring to 1,000 mL of about 80° C.-85° C. water in a glass beaker, preferably a beaker made hydrophobic with dimethyldichlorosilane to reduce the adhesion of PEU to the beaker's walls. The polymer solution is broken in water into small drops and chloroform evaporates rather vigorously. Gradually, as chloroform is evaporated, small drops combine into a compact tar-like mass that is transformed into a sticky rubbery product. This rubbery product is removed from the beaker and put into hydrophobized cylindrical glass-test-tube, which is thermostatically controlled at about 80° C. for about 24 hours. Then the test-tube is removed from the thermostat, cooled to room temperature, and broken to obtain the polymer. The obtained porous bar is placed into a vacuum drier and dried under reduced pressure at about 80° C. for about 24 hours. In addition, any procedure known in the art for obtaining porous polymeric materials can also be used.

Properties of high-molecular-weight porous PEUs made by the above procedure yielded results as summarized in Table 1.

TABLE 1
Properties of PEU Polymers of Formula (VI).

| | Yield | η_{red}^{a)} | | | M_{w}/ | Tg ^{c)} | Tₘ^{c)} |
|---|---|---|---|---|---|---|---|
| PEU* | [%] | [dL/g] | M_{w}^{b)} | Mₙ^{b)} | Mₙ^{b)} | [° C.] | [° C.] |
| 1-L-Leu-4 | 80 | 0.49 | 84000 | 45000 | 1.90 | 67 | 103 |
| 1-L-Leu-6 | 82 | 0.59 | 96700 | 50000 | 1.90 | 64 | 126 |
| 1-L-Phe-6 | 77 | 0.43 | 60400 | 34500 | 1.75 | — | 167 |
| [1-L- | 84 | 0.31 | 64400 | 43000 | 1.47 | 34 | 114 |
| Leu-6]_{0.75}- | | | | | | | |
| [1-L-Lys | | | | | | | |
| (OBn)]_{0.25} | | | | | | | |
| 1-L-Leu- | 57 | 0.28 | 55700^{d)} | 27700^{d)} | 2.1^{d)} | 56 | 165 |
| DAS | | | | | | | |

*In general PEU formula (VI)
1-L-Leu-4 = R¹ = (CH₂)₄, R³ = i-C₄H₉
1-L-Leu-6 = R¹ = (CH₂)₆, R³ = i-C₄H₉
1-L-Phe-6: = .R¹ = (CH₂)₆, R³ = —CH₂—C₆H₅.
1-L-Leu-DAS = R¹ = 1,4:3,6-dianhydrosorbitol, R³ = i-C₄H
^{a)} Reduced viscosities were measured in N,N-dimethylformamide (DMF) at 25° C. and a concentration 0.5 g/dL
^{b)} GPC Measurements were carried out in DMF, (PMMA)
^{c)} Tg taken from second heating curve from DSC Measurements (heating rate 10° C./min).
^{d)}GPC Measurements were carried out in DMAc, (PS)

Tensile strength of illustrative synthesized PEUs was measured and results are summarized in Table 2. Tensile strength measurement was obtained using dumbbell-shaped PEU films (4×1.6 cm), which were cast from chloroform solution with average thickness of 0.125 mm and subjected to tensile testing on tensile strength machine (Chatillon TDC200) integrated with a PC using Nexygen FM software (Amtek, Largo, Fla.) at a crosshead speed of 60 mm/min. Examples illustrated herein can be expected to have the following mechanical properties:

1. A glass transition temperature in the range from about 30° C. to about 90° C., for example, in the range from about 35° C. to about 65° C.;

2. A film of the polymer with average thickness of about 1.6 cm will have tensile stress at yield of about 20 Mpa to about 150 Mpa, for example, about 25 Mpa to about 60 Mpa;

3. A film of the polymer with average thickness of about 1.6 cm will have a percent elongation of about 10% to about 200%, for example about 50% to about 150%; and

4. A film of the polymer with average thickness of about 1.6 cm will have a Young's modulus in the range from about 500 MPa to about 2000 MPa. Table 2 below summarizes the properties of exemplary PEUs of this type.

TABLE 2

| | | Tensile Stress | Percent | Young's |
|---|---|---|---|---|
| | Tg^{a)} | at Yield | Elongation | Modulus |
| Polymer designation | (° C.) | (MPa) | (%) | (MPa) |
| 1-L-Leu-6 | 64 | 21 | 114 | 622 |
| [1-L-Leu-6]_{0.75-} [1-L- | 34 | 25 | 159 | 915 |
| Lys(OBn)]_{0.25} | | | | |

^{a)Tg taken from second heating curve from DSC Measurements (heating rate 10° C. /min).}

Polymers useful in the invention polymer particle delivery compositions, such as PEA, PEUR and PEU polymers, biodegrade by enzymatic action at the surface. Therefore, the polymers, for example particles thereof, administer the macromolecular biologic and any bioactive agent to the subject at a controlled release rate, which is specific and constant over a prolonged period. Additionally, since PEA, PEUR and PEU polymers break down in vivo via hydrolytic enzymes without production of adverse side-products, the invention polymer particle delivery compositions are substantially non-inflammatory.

As used herein “dispersed” means at least one bioactive agent as disclosed herein is dispersed, mixed, dissolved, homogenized, and/or covalently bound (“dispersed”) in a polymer particle, for example attached to the surface of the particle. As used herein to refer to a macro macromolecular molecule, “disbursed” specifically includes, but is not limited to, conjugation of one or more macromolecular biologic or promoter, or oligomer thereof to the polymer.

While the optional bioactive agents can be dispersed within the polymer matrix without chemical linkage to the polymer carrier, it is also contemplated that the bioactive agent or covering molecule, if used, can be covalently bound to the biodegradable polymers via a wide variety of suitable functional groups. For example, when the biodegradable polymer is a polyester, the carboxyl group chain end can be used to react with a complimentary moiety on the bioactive agent or covering molecule, such as hydroxy, amino, thio, and the like. A wide variety of suitable reagents and reaction conditions are disclosed, e.g., in March's Advanced Organic Chemistry, Reactions, Mechanisms, and Structure, Fifth Edition, (2001); and Comprehensive Organic Transformations, Second Edition, Larock (1999).

In other embodiments, a bioactive agent can be linked to the PEA, PEUR or PEU polymers described herein through an amide, ester, ether, amino, ketone, thioether, sulfinyl, sulfonyl, disulfide linkage. Such a linkage can be formed from suitably functionalized starting materials using synthetic procedures that are known in the art.

For example, in one embodiment a polymer can be linked to the bioactive agent via a carboxyl group (e.g., COOH) of the polymer. For example, a compound of structures (I) and (IV) can react with an amino functional group or a hydroxyl functional group of a bioactive agent to provide a biodegradable polymer having the bioactive agent attached via an amide linkage or carboxylic ester linkage, respectively. In another embodiment, the carboxyl group of the polymer can be benzylated or transformed into an acyl halide, acyl anhydride/“mixed” anhydride, or active ester. In other embodiments, the free —NH₂ ends of the polymer molecule can be acylated to assure that the bioactive agent will attach only via a carboxyl group of the polymer and not to the free ends of the polymer.

Water soluble covering molecule(s), such as poly(ethylene glycol) (PEG); phosphoryl choline (PC); glycosaminoglycans including heparin; polysaccharides including polysialic acid; poly(ionizable or polar amino acids) including polyserine, polyglutamic acid, polyaspartic acid, polylysine and polyarginine; chitosan and alginate, as described herein, and targeting molecules, such as antibodies, antigens and ligands, can also be conjugated to the polymer in the exterior of the particles after production of the particles to block active sites not occupied by the bioactive agent or to target delivery of the particles to a specific body site as is known in the art. The molecular weights of PEG molecules on a single particle can be substantially any molecular weight in the range from about 200 to about 200,000, so that the molecular weights of the various PEG molecules attached to the particle can be varied.

Alternatively, the bioactive agent or covering molecule can be attached to the polymer via a linker molecule, for example, as described in structural formulas (VII-XI). Indeed, to improve surface hydrophobicity of the biodegradable polymer, to improve accessibility of the biodegradable polymer towards enzyme activation, and to improve the release profile of the biodegradable polymer, a linker may be utilized to indirectly attach the bioactive agent to the biodegradable polymer. In certain embodiments, the linker compounds include poly(ethylene glycol) having a molecular weight (MW) of about 44 to about 10,000, preferably 44 to 2000; amino acids, such as serine; polypeptides with repeat number from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄)alkyl, (C₂-C₂₄)alkenyl, (C₂-C₂₄)alkynyl, (C₃-C₈)cycloalkyl, or (C₆-C₁₀) aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein R is independently H or (C₁-C₆)alkyl.

As used to describe the above linkers, the term “alkyl” refers to a straight or branched chain hydrocarbon group including methyl, ethyl, n-propyl, isopropyl, n-butyl, isobutyl, tert-butyl, n-hexyl, and the like.

As used to describe the above linkers, “alkenyl” refers to straight or branched chain hydrocarbyl groups having one or more carbon-carbon double bonds.

As used to describe the above linkers, “alkynyl” refers to straight or branched chain hydrocarbyl groups having at least one carbon-carbon triple bond.

As used to describe the above linkers, “aryl” refers to aromatic groups having in the range of 6 up to 14 carbon atoms.

In certain embodiments, the linker may be a polypeptide having from about 2 up to about 25 amino acids. Suitable peptides contemplated for use include poly-L-glycine, poly-L-lysine, poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine, poly-L-arginine, poly-L-lysine-L-tyrosine, and the like.

In one embodiment, the bioactive agent can covalently crosslink the polymer, i.e. the bioactive agent is bound to more than one polymer molecule. This covalent crosslinking can be done with or without additional polymer-bioactive agent linker.

The bioactive agent molecule can also be incorporated into an intramolecular bridge by covalent attachment between two polymer molecules.

A linear polymer polypeptide conjugate is made by protecting the potential nucleophiles on the polypeptide backbone and leaving only one reactive group to be bound to the polymer or polymer linker construct. Deprotection is performed according to methods well known in the art for deprotection of peptides (Boc and Fmoc chemistry for example).

In one embodiment of the present invention, a polypeptide bioactive agent is presented as retro-inverso or partial retro-inverso peptide.

In other embodiments the bioactive agent is mixed with a photocrosslinkable version of the polymer in a matrix, and after crosslinking the material is dispersed (ground) to an average diameter in the range from about 0.1 to about 10 μm.

The linker can be attached first to the polymer or to the bioactive agent or covering molecule. During synthesis, the linker can be either in unprotected form or protected form, using a variety of protecting groups well known to those skilled in the art. In the case of a protected linker, the unprotected end of the linker can first be attached to the polymer or the bioactive agent or covering molecule. The protecting group can then be de-protected using Pd/H₂ hydrogenolysis, mild acid or base hydrolysis, or any other common de-protection method that is known in the art. The de-protected linker can then be attached to the bioactive agent or covering molecule, or to the polymer

An exemplary synthesis of a biodegradable polymer according to the invention (wherein the molecule to be attached is an aminoxyl) is set forth as follows.

A polyester can be reacted with an amino-substituted aminoxyl (N-oxide) radical bearing group, e.g., 4-amino-2,2,6,6-tetramethylpiperidine-1-oxy, in the presence of N,N′-carbonyldiimidazole to replace the hydroxyl moiety in the carboxyl group at the chain end of the polyester with an amino-substituted aminoxyl-(N-oxide) radical bearing group, so that the amino moiety covalently bonds to the carbon of the carbonyl residue of the carboxyl group to form an amide bond. The N,N′-carbonyl diimidazole or suitable carbodiimide converts the hydroxyl moiety in the carboxyl group at the chain end of the polyester into an intermediate product moiety which will react with the aminoxyl, e.g., 4-amino-2,2,6,6-tetramethylpiperidine-1-oxy. The aminoxyl reactant is typically used in a mole ratio of reactant to polyester ranging from 1:1 to 100:1. The mole ratio of N,N′-carbonyl diimidazole to aminoxyl is preferably about 1:1.

A typical reaction is as follows. A polyester is dissolved in a reaction solvent and reaction is readily carried out at the temperature utilized for the dissolving. The reaction solvent may be any in which the polyester will dissolve. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 115° C. to 130° C. or DMSO at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, dichloromethane (DCM) and chloroform at room temperature to 40˜50° C. suitably dissolve the polyester.

Polymer—Bioactive Agent or Macromolecular Biologic Linkage

In one embodiment, the polymers used to make the invention polymer particle delivery compositions as described herein have one or more macromolecular biologic or bioactive agent directly linked to the polymer. The residues of the polymer can be linked to the residues of the one or more macromolecular biologics or bioactive agents. For example, one residue of the polymer can be directly linked to one residue of the macromolecular biologic or bioactive agent. In the case of a macromolecular biologic with more than one open valence, the macromolecular biologic can be directly linked to more than one residue in the polymer. Alternatively, more than one, multiple, or a mixture of macromolecular biologics and bioactive agents having different therapeutic or palliative activity can be directly linked to the polymer. However, since the residue of each macromolecular biologic or bioactive agent can be linked to a corresponding residue of the polymer via at least one point of conjugation, the number of residues of the one or more macromolecular biologic or bioactive agents can correspond to the number of open valences on the residue of the polymer.

The invention compositions and methods encompass the use of RNA and DNA of all types as macromolecular biologics. In one embodiment, the macromolecular biologic is a nucleic acid, oligonucleotide or polynucleotide. More specifically, the nucleic acid is any DNA or RNA. RNA includes messenger (mRNA), transfer (tRNA), ribosomal (rRNA), and interfering (iRNA). Interfering RNA is any RNA involved in post-transcriptional gene silencing, which includes but is not limited to, double stranded RNA (dsRNA), small interfering RNA (siRNA), and microRNA (miRNA) that are comprised of sense and antisense strands. In the mechanism of RNA interference, dsRNA enters a cell and is digested to 21-23 nucleotide siRNAs by the enzyme DICER. Successive cleavage events degrade the RNA to 19-21 nucleotides. The siRNA antisense strand binds a nuclease complex to form the RNA-induced silencing complex, or RISC. Activated RISC targets the homologous transcript by base pairing interactions and cleaves the mRNA, thereby suppressing expression of the target gene. Recent evidence suggests that the machinery is largely identical for miRNA (Cullen, B. R. (2004) Virus Res. 102:3). In this way, iRNA, associated with the polymer, can be delivered into cells by phago- or pino-cytosis and released to enter its normal biological processing pathway.

The emerging sequence-specific inhibitors of gene expression, small interfering RNAs (siRNAs), have great therapeutic potential; however, development of such molecules as therapeutic agents is hampered by rapid degradation of siRNA in vivo. Therefore a key requirement for success in therapeutic use of siRNA is the protection of the gene silencing nucleic acid. In the present invention, such protection to siRNA is provided by conjugation to V or a PEU of structure VI or VII biodegradable polymers described herein, such as PEA, PEUR or PEU molecules described by structural Formulas III, V, and VII, respectively, which provide opportunities for conjugation of RNA (or DNA) using procedures well known in the art.

For, example, in fabrication of the invention particles for delivery of the antisense strand of iRNA, the sense stand of iRNA is conjugated to the polymer active groups by either the 3′ or the 5′ end. The antisense strand is associated with the polymer only through normal base pairing of the nucleotides (i.e., a form of aggregation), the antisense strand being provided in the reaction solution. Alternatively, the sense strand can be conjugated to one polymer chain and the antisense strand to another polymer chain. Base pairing of the strands will stabilize the particles. In either case, additional, non-conjugated RNA can be added to the particle. The double stranded RNA, cleaved from the particle during biodegradation of the particles, or the antisense strand, freed from the sense strand, would enter the normal biological pathway for iRNA.

Examples of such procedures are illustrated schematically below: 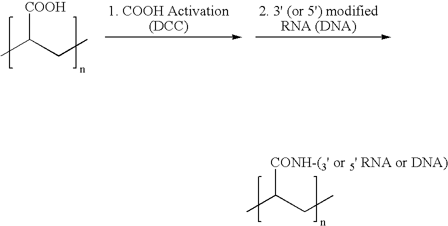
The conjugation of DNA or RNA to PEA, PEUR or PEU can be achieved by, but is not limited to, use of the 3′- or 5′-aminomodifiers shown below:

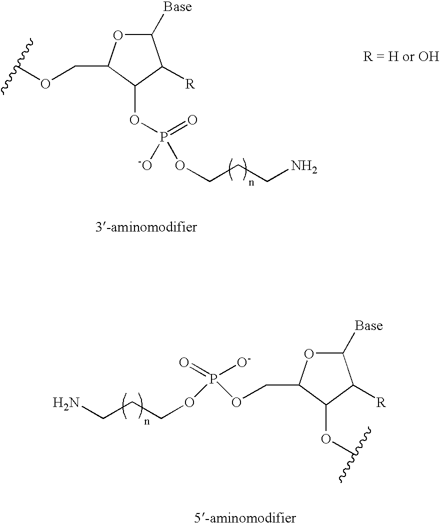

Using such aminomodifiers, those of skill in the art can covalently conjugate an oligonucleotide to the polymer through the amide bond therein. Alternatively, a suitable bifunctional linker such as is described herein can be incorporated between the polymer and the nucleic acids. In a similar way other biologically active molecules, such as lipids and mono- and polysaccharides can be conjugated to PEA, PEUR and PEU polymers.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is attached to a residue of a bioactive agent. Additionally, any synthetically feasible functional group (e.g., carboxyl) can be created on the polymer (e.g., on the polymer backbone or pendant group) to provide the open valence, provided bioactivity is substantially retained when the radical is attached to a residue of a bioactive agent. Based on the linkage that is desired, those skilled in the art can select suitably functionalized starting materials that can be derived from the polymer of the present invention using procedures that are known in the art.

As used herein, a “residue of a compound of structural formula (*)” refers to a radical of a compound of polymer formulas (I) and (III-VII) as described herein having one or more open valences. Any synthetically feasible atom, atoms, or functional group of the compound (e.g., on the polymer backbone or pendant group) can be removed to provide the open valence, provided bioactivity is substantially retained when the radical is attached to a residue of an bioactive agent. Additionally, any synthetically feasible functional group (e.g., carboxyl) can be created on the compound of formulas (I) and (III-VII) (e.g., on the polymer backbone or pendant group) to provide the open valance, provided bioactivity is substantially retained when the radical is attached to a residue of a bioactive agent. Based on the linkage that is desired, those skilled in the art can select suitably functionalized starting materials that can be derived from the compound of formulas (I) and III-VII) using procedures that are known in the art.

For example, the residue of a bioactive agent can be linked to the residue of a compound of structural formula (I) or (III) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein R is independently H or (C₁-C₆) alkyl. Such a linkage can be formed from suitably functionalized starting materials using synthetic procedures that are known in the art. Based on the linkage that is desired, those skilled in the art can select suitably functional starting material that can be derived from a residue of a compound of structural formula (I) or (III) and from a given residue of a bioactive agent or adjuvant using procedures that are known in the art. The residue of the bioactive agent or adjuvant can be linked to any synthetically feasible position on the residue of a compound of structural formula (I) or (III). Additionally, the invention also provides compounds having more than one residue of a bioactive agent or adjuvant bioactive agent directly linked to a compound of structural formula (I) or (III).

The number of macromolecular biologic and bioactive agents that can be linked to the polymer molecule can typically depend upon the molecular weight of the polymer and the equivalents of functional groups incorporated. For example, for a compound of structural formula (I), wherein n is about 5 to about 150, preferably about 5 to about 70, up to about 150 macromolecular biologic or bioactive agent molecules (i.e., residues thereof) can be directly linked to the polymer (i.e., residue thereof by reacting the bioactive agent with side groups of the polymer. In unsaturated polymers, the bioactive agents can also be reacted with double (or triple) bonds in the polymer.

The number of macromolecular biologics and bioactive agents that can be linked to the polymer molecule can typically depend upon the molecular weight of the polymer. For example, for a saturated compound of structural formula (I), wherein n is about 5 to about 150, preferably about 5 to about 70, up to about 150 bioactive agents (i.e., residues thereof) can be directly linked to the polymer (i.e., residue thereof) by reacting the bioactive agent with side groups of the polymer. In unsaturated polymers, the bioactive agents can also be reacted with double (or triple) bonds in the polymer.

PEA-, PEUR and PEU polymers described herein minimally absorb water, therefore allowing small hydrophilic molecules to diffuse through hydrophilic surface channels. This characteristic makes these polymers suitable for use as an over coating on particles to regulate controlled release of such molecules. Water absorption also enhances biocompatibility of the polymers and of the polymer particle delivery composition based on such polymers. In addition, due to the partial hydrophilic properties of the PEA, PEUR and PEU polymers, they have a tendency to become sticky and agglomerate, when delivered in vivo as particles at body temperature. Thus the polymer particles spontaneously form polymer depots when injected subcutaneously or intramuscularly for local delivery, such as by subcutaneous needle or needle-less injection. Particles having an average diameter range from about 1 micron to about 500 microns, which size will not circulate efficiently within the body, are suitable for forming such polymer depots in vivo. Alternatively, for oral administration the GI tract can tolerate a much wider range of particle sizes, for example nanoparticles of about 20 nanometers up to micro particles of about 1000 microns average diameter.

Methods for Encapsulation of Macromolecular Biologics within Particles

Although not soluble in water, the types of PEAs, PEURs and PEUs described herein can be solubilized in strong organic solvents such as dichloromethane (DCM) or dimethylsulfoxide (DMSO), as well as in highly polar fluorinated solvents such as hexafluoroisopropanol (HFIP) and tetrafluoroethylene (TFE). These two solvent types lead to two quite different encapsulation techniques, both however based upon the emulsification of immiscible solvents. It is important to note that, unlike for example ethanol, both of these types of solvent are non-dehydrating and need not destabilize bound water. Moreover, significant doping of these strong organic solvents with additional water molecules is possible, along with other ionic enhancers of biologic stability and assembly, such as metal ions and surfactants, to enhance the encapsulation of macromolecular biologics within the polymer particles used in the invention compositions and methods.

Encapsulation Method 1: water in organic solvent (w/o emulsion) Surprisingly, while the structural fold of most macromolecular biologics is not stable in strong organic solvents, such as DCM; small crystals of a very few macromolecular biologics, such as Zn-insulin, are stable in strong organic solvents. The following steps can be used to encapsulate small crystals of macromolecular biologics, such as Zn-insulin, that are stable in strong organic solvents.

Nano-/micro-crystals of Zn-insulin are prepared by micro-titration of Zn-insulin between a soluble phase and an insoluble phase, in such a way as to preserve the bound water of crystallization therein.

The crystals are mixed with a polymer, such as PEA in DCM, in the presence of surfactant-A to form a liquid-solid slurry. This liquid-solid slurry, containing a small fraction of water, is emulsified in bulk water containing surfactant-B. The energy of emulsification is provided by a procedure of vortexing, followed by sonication, followed by again vortexing. Phase separation occurs at the water/organic interface so that the polymer wraps the crystalline Zn-insulin into particles.

The volatile organic phase is removed by rotary evaporation, and, importantly, this procedure is not driven to complete dryness to allow the non-volatile residual water to remain with the Zn-insulin in the particles. The particle aggregate so formed can be re-dispersed in water containing surfactant-C.

Such a dispersion of particles optionally can be lyophilized to a powder of polymer particles containing micro-crystalline Zn-insulin and bound water for ease of transportation and storage. The lyophilized particles can be re-constituted in a suitable medium for administration, as described herein and as is known in the art.

Encapsulation Method 2: oil organic in non-polar organic (o/o) Although this method is illustrated with insulin, it is applicable to macromolecular biologics in general. The insulin monomer is small and strongly stabilized by covalent disulphide bonds. By contrast, most proteins are larger than and not as inherently stable as insulin.

Zn-insulin is dissolved with PEA or PEUR in warm HFIP/TFE. (In general, other molecules such as salts, ions and/or biologically compatible surfactants, as are known to those of skill in the art can be added so as to promote the stabilization of the biologic by micro-crystallization during stage (iii) below):

The polymer-biologic mixture is emulsified in bulk cotton-seed oil containing surfactant-D. The energy of emulsification is provided by mixing at high rpms, and phase separation occurs at the o/o interface so that the polymer wraps the inner polar organic phase, containing the Zn-insulin, into particles.

The oil organic phase is then removed by washing in hexane over a vacuum-filter, and volatile solvents (hexane, HFIP, TFE) are removed by lyophilization. Importantly this procedure allows the non-volatile bound water to remain with the Zn-insulin, promoting crystallization of insulin oligomers within the shrinking polar interior of the particles.

The resulting particle aggregate is re-dispersed in water containing surfactant-E. Although surfactants A-E may be selected by those skilled in the art for their ability to solubilize the particular molecule(s) at hand, there may be occasions when surfactants A-E will be selected from a small number of biologically compatible surfactants, e.g. one, two, or three biologically compatible surfactants will suffice for surfactants A-E. This dispersion optionally can be re-lyophilized to a powder of polymer particles containing crystalline Zn-insulin and bound water.

The aim of these methods is to stabilize the biologic by promoting interactions both with itself and with the wrapping polymer. To achieve this with most biologics a mixture of both hydrophobic and ionic interactions is important, and the appropriate strength of the ionic bonds is particularly important.

The Examples contained herein demonstrate that the inclusion of the free-COOH CO-polymer version in step (ii) enhances both loading and stability of Zn-insulin compared with un-charged polymers. This is presumably because of local charge interactions between the —COOH and primary amines on the biologic, or with zinc. In addition, the Examples contained herein demonstrate that loading and stability can be further enhanced by the replacement of Zn-insulin in step (i) with a formulation of Zn-insulin-PEA, pre-prepared as follows:

Method for the Seeding of Biologic Oligomerization and Crystallization by Polymer-Biologic Conjugates

Here monomers of the macromolecular biologic, illustrated here by free insulin, are conjugated to PEA-H (Formula III; R²=H) by the methods described herein and as are known in the art. For insulin, an A and a B chain form one promoter, in which the two chains are linked together covalently by two disulphide bonds. Conjugation to the polymer by amide bond formation can be to either, or both, chains of the insulin promoter.

Free insulin is then added in the presence of Zn and in conditions that promote oligomerization and crystallization. It is envisioned that oligomerization stabilizes the re-folding of the insulin conjugate in the presence of five additional monomers. In some cases we can expect a percentage of polymer chains will be cross-linked by this hexamerization, in which the hexamer contains more than one conjugate, but in general there will be one conjugated insulin monomer per Zn-hexamer. The percentage of cross-linking will also depend upon such factors as the density of loading of insulin the amount of conjugate per polymer chain, and upon the relative amounts of conjugate to free insulin. These fixed Zn-hexamers seed the crystallization of adjacent excess free Zn-hexamers around them.

The whole mixture of conjugate and free insulin is concentrated by lyophilization, resulting in a powder containing up to 95% free insulin which nonetheless is significantly protected and strengthened during subsequent processing steps by the presence of the polymer.

General Application of these Methods

Although insulin has been used as the example of a macromolecular biologic to illustrate the invention, in principle the compositions and methods described herein are applicable to the preservation and delivery of any macromolecule. The key feature is the use of the peculiarities of amino acid based polymers to enhance the stability of micro-condensations of macromolecules. These micro-condensates can include true crystalline, or partially crystalline arrays, either oligomeric or monomeric.

In principle, any macromolecule can be protected and delivered by this method.

Synthetic vaccine preparations can also be improved by this type of formulation, in which antigen structure is preserved, thus allowing antibody recognition, leading to enhancement of B-cell as well as T-cell responses.

Particles suitable for use in the invention polymer particle delivery compositions can be made using immiscible solvent techniques. Generally, these methods entail the preparation of an emulsion of two immiscible liquids. A single emulsion method can be used to make polymer particles that incorporate at least one hydrophobic bioactive agent. In the single emulsion method, bioactive agents to be incorporated into the particles are mixed with polymer in solvent first, and then emulsified in water solution with a surface stabilizer, such as a surfactant. In this way, polymer particles with hydrophobic bioactive agent conjugates are formed and suspended in the water solution, in which hydrophobic conjugates in the particles will be stable without significant elution into the aqueous solution, but such molecules will elute into body tissue, such as muscle tissue.

Most biologics, including polypeptides, proteins, DNA, cells and the like, are hydrophilic. A double emulsion method can be used to make polymer particles with interior aqueous phase and hydrophilic optional bioactive agents dispersed within. In the double emulsion method, aqueous phase or hydrophilic bioactive agents dissolved in water are emulsified in polymer lipophilic solution first to form a primary emulsion, and then the primary emulsion is put into water to emulsify again to form a second emulsion, in which particles are formed having a continuous polymer phase and aqueous macromolecular biologic in the dispersed phase.

Surfactant and additive can be used in both emulsifications to prevent particle aggregation. Chloroform or DCM, which are not miscible in water, are used as solvents for PEA and PEUR polymers, but later in the preparation the solvent is removed, using methods known in the art.

For certain bioactive agents with low water solubility, however, these two emulsion methods have limitations. In this context, “low water solubility” means a bioactive agent that is less hydrophobic than truly lipophilic drugs, such as Taxol, but which are less hydrophilic than truly water-soluble drugs, such as many biologics. These types of intermediate compounds are too hydrophilic for high loading and stable matrixing into single emulsion particles, yet are too hydrophobic for high loading and stability within double emulsions. In such cases, a polymer layer is coated onto particles made of polymer and drugs with low water solubility, by a triple emulsion process, as illustrated schematically in FIG. 7. This method provides relatively low drug loading (˜10% w/w), but provides structure stability and controlled drug release rate.

In the triple emulsion process, the first emulsion is made by mixing the bioactive agents into polymer solution and then emulsifying the mixture in aqueous solution with surfactant or lipid, such as di-(hexadecanoyl)phosphatidylcholine (DHPC; a short-chain derivative of a natural lipid). In this way, particles containing the active agents are formed and suspended in water to form the first emulsion. The second emulsion is formed by putting the first emulsion into a polymer solution, and emulsifying the mixture, so that water drops with the polymer/drug particles inside are formed within the polymer solution. Water and surfactant or lipid will separate the particles and dissolve the particles in the polymer solution. The third emulsion is then formed by putting the second emulsion into water with surfactant or lipid, and emulsifying the mixture to form the final particles in water. The resulting particle structure, as illustrated in FIG. 7, will have one or more particles made with polymer plus bioactive agent at the center, surrounded by water and surface stabilizer, such as surfactant or lipid, and covered with a pure polymer shell. Surface stabilizer and water will prevent solvent in the polymer coating from contacting the particles inside the coating and dissolving them.

To increase loading of bioactive agents by the triple emulsion method, active agents with low water solubility can be coated with surface stabilizer in the first emulsion, without polymer coating and without dissolving the bioactive agent in water. In this first emulsion, water, surface stabilizer and active agent have similar volume or in the volume ratio range of (1 to 3):(0.2 to about 2): 1, respectively. In this case, water is used, not for dissolving the active agent, but rather for protecting the bioactive agent with help of surface stabilizer. Then the double and triple emulsions are prepared as described above. This method can provide up to 50% drug loading.

Alternatively or additionally in the single, double or triple emulsion methods described above, a bioactive agent or macromolecular biologic can be conjugated to the polymer molecule as described herein prior to using the polymers to make the particles. Alternatively still, a bioactive agent or macromolecular biologic can be conjugated to the polymer on the exterior of the particles described herein after production of the particles.

Many emulsification techniques will work in making the emulsions described above. However, the presently preferred method of making the emulsion is by using a solvent that is not miscible in water. For example, in the single emulsion method, the emulsifying procedure consists of dissolving polymer with the solvent, mixing with macromolecular biologic and/or bioactive agent molecule(s), putting into water, and then stirring with a mixer and/or ultra-sonicator. Particle size can be controlled by controlling stir speed and/or the concentration of polymer, bioactive agent(s), and surface stabilizer. Coating thickness, if a coating is used, can be controlled by adjusting the ratio of the second to the third emulsion.

Suitable emulsion stabilizers may include nonionic surface active agents, such as mannide monooleate, dextran 70,000, polyoxyethylene ethers, polyglycol ethers, and the like, all readily commercially available from, e.g., Sigma Chemical Co., St. Louis, Mo. The surface active agent will be present at a concentration of about 0.3% to about 10%, preferably about 0.5% to about 8%, and more preferably about 1% to about 5%.

Rate of release of the at least one macromolecular biologic from the invention particle delivery compositions can be controlled by adjusting the coating thickness, particle size, structure, and density of the coating. Density of the coating can be adjusted by adjusting loading of the bioactive agent conjugated to the coating. For example, when the coating contains no bioactive agent, the polymer coating is densest, and a macromolecular biologic or bioactive agent from the interior of the particle elutes through the coating most slowly. By contrast, when a bioactive agent is loaded into (i.e. is mixed or “matrixed” with), or alternatively is conjugated to, polymer in the coating, the coating becomes porous once the bioactive agent has become free of polymer and has eluted out, starting from the outer surface of the coating. Thereby, a macromolecular biologic or optional bioactive agent at the center of the particle can elute at an increased rate. The higher the loading in the coating, the lower the density of the coating layer and the higher the elution rate. The loading of bioactive agent in the coating can be lower or higher than that of the macromolecular biologic in the interior of the particles beneath the exterior coating. Release rate of macromolecular biologics and/or bioactive agent(s) from the particles can also be controlled by mixing particles with different release rates prepared as described above.

A detailed description of methods of making double and triple emulsion polymers may be found in Pierre Autant et al, Medicinal and/or nutritional microcapsules for oral administration, U.S. Pat. No. 6,022,562; Iosif Daniel Rosca et al., Microparticle formation and its mechanism in single and double emulsion solvent evaporation, Journal of Controlled Release 99 (2004) 271-280; L. Mu, S. S. Feng, A novel controlled release formulation for the anticancer drug paclitaxel (Taxol): PLGA nanoparticles containing vitamin E TPGS, J. Control. Release 86 (2003) 33-48; Somatosin containing biodegradable microspheres prepared by a modified solvent evaporation method based on W/O/W-multiple emulsions, Int. J. Pharm. 126 (1995) 129-138 and F. Gabor, B. Ertl, M. Wirth, R. Mallinger, Ketoprofenpoly(d,l-lactic-co-glycolic acid) microspheres: influence of manufacturing parameters and type of polymer on the release characteristics, J. Microencapsul. 16 (1) (1999) 1-12, each of which is incorporated herein in its entirety.

In yet further embodiments for delivery of the macromolecular biologics and optional aqueous-soluble bioactive agents, the particles can be made into nanoparticles having an average diameter of about 20 nm to about 200 nm for delivery to the circulation. The nanoparticles can be made by the single emulsion method with the macromolecular biologic dispersed therein, i.e., mixed into the emulsion or conjugated to polymer as described herein. The nanoparticles can also be provided as a micellar composition containing the PEA, PEUR and PEU polymers described herein with the bioactive agents conjugated thereto. Since the micelles are formed in water, optionally water soluble bioactive agents can be loaded into the micelles at the same time without solvent.

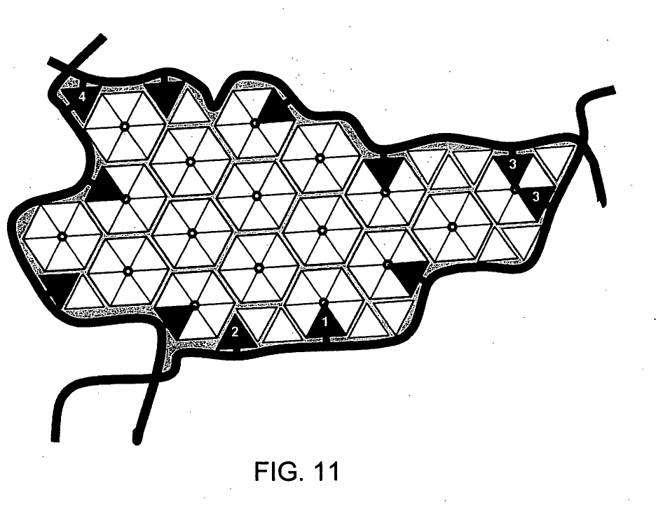

More particularly, the biodegradable micelles, which are illustrated in FIG. 10, are formed of a hydrophobic polymer chain conjugated to a water soluble polymer chain. Whereas, the outer portion of the micelle mainly consists of the water soluble ionized or polar section of the polymer, the hydrophobic section of the polymer mainly partitions to the interior of the micelles and holds the polymer molecules together.

The biodegradable hydrophobic section of the polymer is made of PEA. PEUR or PEU polymers, as described herein. For strongly hydrophobic PEA, PEUR or PEU segments, components such as carboxylate phenoxy propene (CPP) and/or leucine-1,4:3,6-dianhydro-D-sorbitol (DAS) may be included in the polymer repeat unit. By contrast, the water soluble section of the polymer comprises repeating alternating units of polyethylene glycol, polyglycosaminoglycan or polysaccharide and at least one ionizable or polar amino acid, wherein the repeating alternating units have substantially similar molecular weights and wherein the molecular weight of the polymer is in the range from about 10 kD to about 300 kD. The repeating alternating units may have substantially similar molecular weights in the range from about 300 D to about 700 D. In one embodiment wherein the molecular weight of the polymer is over 10 kD, at least one of the amino acid units is an ionizable or polar amino acid selected from serine, glutamic acid, aspartic acid, lysine and arginine. In one embodiment, the units of ionizable amino acids comprise at least one block of ionizable poly(amino acids), such as glutamate or aspartate, can be included in the polymer. The invention micellar composition may further comprise a pharmaceutically acceptable aqueous media with a pH value at which at least a portion of the ionizable amino acids in the water soluble sections of the polymer are ionized.

The higher the molecular weight of the water soluble section of the polymer, the greater the porosity of the micelle and the higher the loading into the micelles of macromolecular biologics and water soluble bioactive agents. In one embodiment, therefore, the molecular weight of the complete water soluble section of the polymer is in the range from about 5 kD to about 100 kD.

Once formed, the micelles can be lyophilized for storage and shipping and reconstituted in the above-described aqueous media. However, it is not recommended to lyophilize micelles containing macromolecular biologics or bioactive agents, such as certain proteins, that would be denatured by the lyophilization process.

Charged moieties within the micelles partially separate from each other in water, and create space for absorption of water soluble macromolecular biologics and optional water soluble bioactive agent(s). Ionized chains with the same type of charge will repel each other and create more space. The ionized polymer also attracts the macromolecular biologic, providing stability to the matrix. In addition, the water soluble exterior of the micelle prevents adhesion of the micelles to proteins in body fluids after ionized sites are taken by the macromolecular biologics and optional bioactive agent. This type of micelle has very high porosity, up to 95% of the micelle volume, allowing for high loading of aqueous-soluble macromolecular biologics and additional aqueous soluble bioactive agents such as polypeptides, DNA, and other bioactive agents. Particle size range of the micelles is about 20 nm to about 200 nm, with about 20 nm to about 100 nm being preferred for circulation in the blood.

Particle size can be determined by, e.g., laser light scattering, using for example, a spectrometer incorporating a helium-neon laser. Generally, particle size is determined at room temperature and involves multiple analyses of the sample in question (e.g., 5-10 times) to yield an average value for the particle diameter. Particle size is also readily determined using scanning electron microscopy (SEM). In order to do so, dry particles are sputter-coated with a gold/palladium mixture to a thickness of approximately 100 Angstroms, and then examined using a scanning electron microscope. Alternatively, the polymer, either in the form of particles or not, can be covalently attached directly to the macromolecular biologic, or at least one promoter thereof, using any of several methods well known in the art and as described hereinbelow. The macromolecular biologic content is generally in an amount that represents approximately 0.1% to about 40% (w/w) bioactive agent to polymer, more preferably about 1% to about 25% (w/w) bioactive agent, and even more preferably about 2% to about 20% (w/w) bioactive agent. The percentage of macromolecular biologic can depend on the desired dose and the condition being treated, as discussed in more detail below.

Bioactive agents for dispersion into and release from the invention biodegradable polymer particle delivery compositions also include anti-proliferants, rapamycin and any of its analogs or derivatives, paclitaxel or any of its taxene analogs or derivatives, everolimus, Sirolimus, tacrolimus, or any of its -limus named family of drugs, and statins such as simvastatin, atorvastatin, fluvastatin, pravastatin, lovastatin, rosuvastatin, geldanamycins, such as 17AAG (17-allylamino-17-demethoxygeldanamycin); Epothilone D and other epothilones, 17-dimethylaminoethylamino-17-demethoxy-geldanamycin and other polyketide inhibitors of heat shock protein 90 (Hsp90), Cilostazol, and the like.

Further, bioactive agents contemplated for dispersion within the polymers used in the invention polymer particle delivery compositions include agents that, when freed or eluted from the polymer particles during their degradation, promote endogenous production of a therapeutic natural wound healing agent, such as nitric oxide, which is endogenously produced by endothelial cells. Alternatively the bioactive agents released from the polymers during degradation may be directly active in promoting natural wound healing processes by endothelial cells. These bioactive agents can be any agent that donates, transfers, or releases nitric oxide, elevates endogenous levels of nitric oxide, stimulates endogenous synthesis of nitric oxide, or serves as a substrate for nitric oxide synthase or that inhibits proliferation of smooth muscle cells. Such agents include, for example, aminoxyls, furoxans, nitrosothiols, nitrates and anthocyanins; nucleosides such as adenosine and nucleotides such as adenosine diphosphate (ADP) and adenosine triphosphate (ATP); neurotransmitter/neuromodulators such as acetylcholine and 5-hydroxytryptamine (serotonin/5-HT); histamine and catecholamines such as adrenalin and noradrenaline; lipid molecules such as sphingosine-1-phosphate and lysophosphatidic acid; amino acids such as arginine and lysine; peptides such as the bradykinins, substance P and calcium gene-related peptide (CGRP), and proteins such as insulin, vascular endothelial growth factor (VEGF), and thrombin.

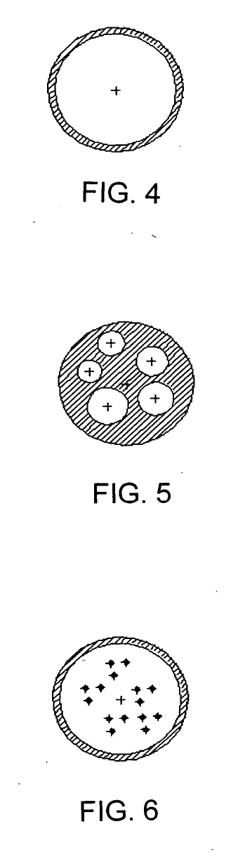

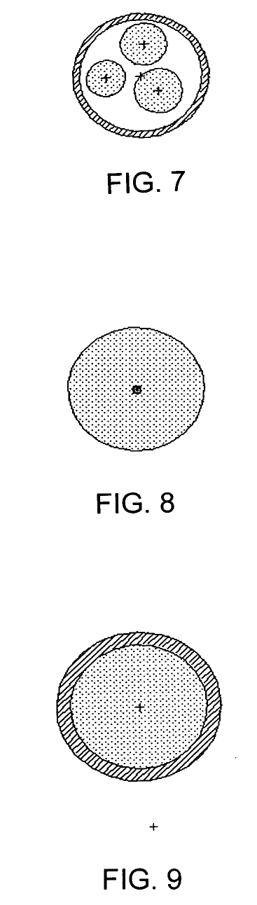

As illustrated in FIG. 2, a variety of bioactive agents, coating molecules and ligands for bioactive agents can be attached, for example covalently, to the surface of the polymer particles. Additional macromolecular biologics and bioactive agents, such as targeting polypeptides (e.g., antigens) and drugs, and the like, can be covalently conjugated to the surface of the polymer particles. In addition, coating molecules, such as polyethylene glycol (PEG) as a ligand for attachment of antibodies or polypeptides or phosphatidylcholine (PC) as a means of blocking attachment sites on the surface of the particles to prevent the particles from sticking to non-target biological molecules and surfaces in the patient may also be surface-conjugated (FIG. 3).

For example, small proteinaceous motifs, such as the B domain of bacterial Protein A and the functionally equivalent region of Protein G are known to bind to, and thereby capture, antibody molecules by the Fc region. Such proteinaceous motifs can be attached to the polymers, especially to the surface of the polymer particles. Such molecules will act, for example, as ligands to attach antibodies for use as targeting ligands or to capture antibodies to hold precursor cells or capture cells out of the patient's blood stream. Therefore, the antibody types that can be attached to polymer coatings using a Protein A or Protein G functional region are those that contain an Fc region. The capture antibodies will in turn bind to and hold precursor cells, such as progenitor cells, near the polymer surface while the precursor cells, which are preferably bathed in a growth medium within the polymer, secrete various factors and interact with other cells of the subject. Optionally, one or more bioactive agents dispersed in the polymer particles, such as the bradykinins, may activate the precursor cells.

The additional macromolecular biologics contemplated for attaching precursor cells or for capturing progenitor endothelial cells (PECs) from the subject's blood include monoclonal antibodies directed against a known precursor cell surface marker. For example, complementary determinants (CDs) that have been reported to decorate the surface of endothelial cells include CD31, CD34, CD102, CD105, CD106, CD109, CDw130, CD141, CD142, CD143, CD144, CDw145, CD146, CD147, and CD166. These cell surface markers can be of varying specificity and the degree of specificity for a particular cell/developmental type/stage is in many cases not fully characterized. In addition these cell marker molecules against which antibodies have been raised will overlap (in terms of antibody recognition) especially with CDs on cells of the same lineage: monocytes in the case of endothelial cells. Circulating endothelial progenitor cells are some way along the developmental pathway from (bone marrow) monocytes to mature endothelial cells. CDs 106, 142 and 144 have been reported to mark mature endothelial cells with some specificity. CD34 is presently known to be specific for progenitor endothelial cells and therefore is currently preferred for capturing progenitor endothelial cells out of blood in the site into which the polymer particles are implanted for local delivery of the active agents. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies.

Due to the versatility of the PEA, PEUR and PEU polymers used in the invention compositions, the amount of the therapeutic diol incorporated in the polymer backbone can be controlled by varying the proportions of the building blocks of the polymer. For example, depending on the composition of the PEA, loading of up to 40% w/w of 17β-estradiol can be achieved. Two different regular, linear PEAs with various loading ratios of 17β-estradiol are illustrated in Scheme 3 below:

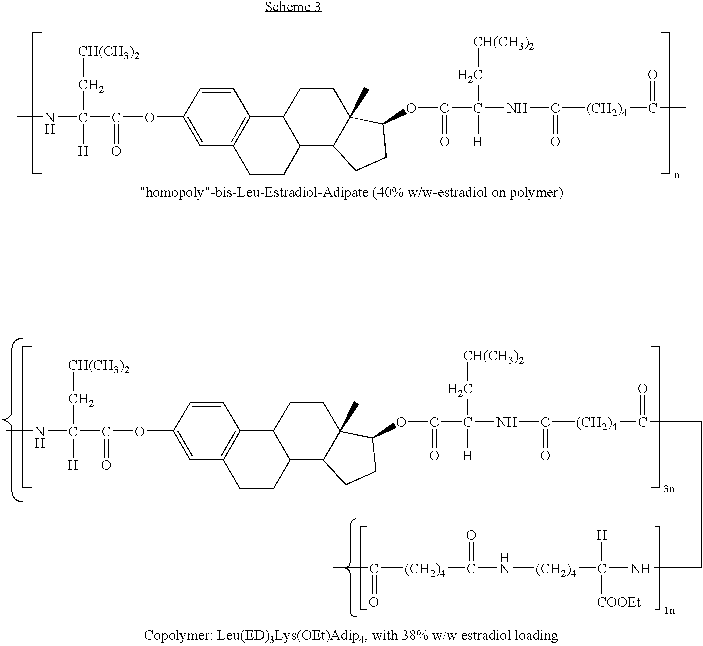

Similarly, the loading of the therapeutic diol into PEUR and PEU polymer can be varied by varying the amount of two or more building blocks of the polymer. Synthesis of a PEUR containing 17-beta-estradiol is illustrated in Example 9 below.

In addition, synthetic steroid based diols based on testosterone or cholesterol, such as 4-androstene-3, 17 diol (4-Androstenediol), 5-androstene-3, 17 diol (5-Androstenediol), 19-nor5-androstene-3, 17 diol (19-Norandrostenediol) are suitable for incorporation into the backbone of PEA and PEUR polymers according to this invention. Moreover, therapeutic diol compounds suitable for use in preparation of the invention polymer particle delivery compositions include, for example, amikacin; amphotericin B; apicycline; apramycin; arbekacin; azidamfenicol; bambermycin(s); butirosin; carbomycin; cefpiramide; chloramphenicol; chlortetracycline; clindamycin; clomocycline; demeclocycline; diathymosulfone; dibekacin, dihydrostreptomycin; dirithromycin; doxycycline; erythromycin; fortimicin(s); gentamycin(s); glucosulfone solasulfone; guamecycline; isepamicin; josamycin; kanamycin(s); leucomycin(s); lincomycin; lucensomycin; lymecycline; meclocycline; methacycline; micronomycin; midecamycin(s); minocycline; mupirocin; natamycin; neomycin; netilmicin; oleandomycin; oxytetracycline; paromycin; pipacycline; podophyllinic acid 2-ethylhydrazine; primycin; ribostamycin; rifamide; rifampin; rafamycin SV; rifapentine; rifaximin; ristocetin; rokitamycin; rolitetracycline; rasaramycin; roxithromycin; sancycline; sisomicin; spectinomycin; spiramycin; streptomycin; teicoplanin; tetracycline; thiamphenicol; theiostrepton; tobramycin; trospectomycin; tuberactinomycin; vancomycin; candicidin(s); chlorphenesin; dermostatin(s); filipin; fungichromin; kanamycin(s); leucomycins(s); lincomycin; lvcensomycin; lymecycline; meclocycline; methacycline; micronomycin; midecamycin(s); minocycline; mupirocin; natamycin; neomycin; netilmicin; oleandomycin; oxytetracycline; paramomycin; pipacycline; podophyllinic acid 2-ethylhydrazine; priycin; ribostamydin; rifamide; rifampin; rifamycin SV; rifapentine; rifaximin; ristocetin; rokitamycin; rolitetracycline; rosaramycin; roxithromycin; sancycline; sisomicin; spectinomycin; spiramycin; strepton; otbramycin; trospectomycin; tuberactinomycin; vancomycin; candicidin(s); chlorphenesin; dermostatin(s); filipin; fungichromin; meparticin; mystatin; oligomycin(s); erimycinA; tubercidin; 6-azauridine; aclacinomycin(s); ancitabine; anthramycin; azacitadine; bleomycin(s) carubicin; carzinophillin A; chlorozotocin; chromomcin(s); doxifluridine; enocitabine; epirubicin; gemcitabine; mannomustine; menogaril; atorvasi pravastatin; clarithromycin; leuproline; paclitaxel; mitobronitol; mitolactol; mopidamol; nogalamycin; olivomycin(s); peplomycin; pirarubicin; prednimustine; puromycin; ranimustine; tubercidin; vinesine; zorubicin; coumetarol; dicoumarol; ethyl biscoumacetate; ethylidine dicoumarol; iloprost; taprostene; tioclomarol; amiprilose; romurtide; sirolimus (rapamycin); tacrolimus; salicyl alcohol; bromosaligenin; ditazol; fepradinol; gentisic acid; glucamethacin; olsalazine; S-adenosylmethionine; azithromycin; salmeterol; budesonide; albuteal; indinavir; fluvastatin; streptozocin; doxorubicin; daunorubicin; plicamycin; idarubicin; pentostatin; metoxantrone; cytarabine; fludarabine phosphate; floxuridine; cladriine; capecitabien; docetaxel; etoposide; topotecan; vinblastine; teniposide, and the like. The therapeutic diol can be selected to be either a saturated or an unsaturated diol.

The following bioactive agents and small molecule drugs optionally can be effectively dispersed within the invention polymer particle compositions, whether sized to form a time release biodegradable polymer depot for local delivery of the macromolecular biologic, or sized for entry into systemic circulation, as described herein. The optional bioactive agents that are dispersed in the polymer particles used in the invention delivery compositions and methods of treatment will be selected for their suitable therapeutic or palliative effect in treatment of a disease of interest, or symptoms thereof.

In one embodiment, the suitable bioactive agents are not limited to, but include, various classes of compounds that facilitate or contribute to wound healing when presented in a time-release fashion. Such bioactive agents include wound-healing cells, including certain precursor cells, which can be protected and delivered by the biodegradable polymer particles in the invention compositions. Such wound healing cells include, for example, pericytes and endothelial cells, as well as inflammatory healing cells. To recruit such cells to the site of a polymer depot in vivo, the polymer particles used in the invention delivery compositions and methods of treatment can include ligands for such cells, such as antibodies and smaller molecule ligands, that specifically bind to “cellular adhesion molecules” (CAMs). Exemplary ligands for wound healing cells include those that specifically bind to Intercellular adhesion molecules (ICAMs), such as ICAM-1 (CD54 antigen); ICAM-2 (CD102 antigen); ICAM-3 (CD50 antigen); ICAM-4 (CD242 antigen); and ICAM-5; Vascular cell adhesion molecules (VCAMs), such as VCAM-1 (CD106 antigen)]; Neural cell adhesion molecules (NCAMs), such as NCAM-1 (CD56 antigen); or NCAM-2; Platelet endothelial cell adhesion molecules PECAMs, such as PECAM-1 (CD31 antigen); Leukocyte-endothelial cell adhesion molecules (ELAMs), such as LECAM-1; or LECAM-2 (CD62E antigen), and the like.].

In another aspect, the suitable bioactive agents include extra cellular matrix proteins, macromolecules that can be dispersed into the polymer particles used in the invention delivery compositions, e.g., attached either covalently or non-covalently. Examples of useful extra-cellular matrix proteins include, for example, glycosaminoglycans, usually linked to proteins (proteoglycans), and fibrous proteins (e.g., collagen; elastin; fibronectins and laminin). Bio-mimics of extra-cellular proteins can also be used. These are usually non-human, but biocompatible, glycoproteins, such as alginates and chitin derivatives. Wound healing peptides that are specific fragments of such extra-cellular matrix proteins and/or their bio-mimics can also be used as the bioactive agent.

Proteinaceous growth factors are another category of bioactive agents that optionally can be dispersed within in the polymer particles used in the invention delivery compositions and methods for delivery of a macromolecular biologic described herein. Such bioactive agents are effective in promoting wound healing and other disease states as is known in the art. For example, Platelet Derived Growth Factor-BB (PDGF-BB), Tumor Necrosis Factor-alpha (TNF-α), Epidermal Growth Factor (EGF), Keratinocyte Growth Factor (KGF), Thymosin B4; and, various angiogenic factors such as vascular Endothelial Growth Factors (VEGFs), Fibroblast Growth Factors (FGFs), Tumor Necrosis Factor-beta (TNF-beta), and Insulin-like Growth Factor-1 (IGF-1). Many of these proteinaceous growth factors are available commercially or can be produced recombinantly using techniques well known in the art.

Alternatively, expression systems comprising vectors, particularly adenovirus vectors, incorporating genes encoding a variety of biomolecules can be dispersed in the polymer particles for timed release delivery. Method of preparing such expression systems and vector are well known in the art. For example, proteinaceous growth factors can be dispersed into the invention polymer particles for administration of the growth factors either to a desired body site for local delivery by selection of particles sized to form a polymer depot or systemically by selection of particles of a size that will enter the circulation. The growth factors such as VEGFs, PDGFs, FGF, NGF, and evolutionary and functionally related biologics, and angiogenic enzymes, such as thrombin, may also be used as bioactive agents in the invention.

Small molecule drugs are yet another category of bioactive agents that optionally can be dispersed in the polymer particles used in the invention delivery compositions and methods for delivery of a macromolecular biologic described herein. Such drugs include, for example, antimicrobials and anti-inflammatory agents as well as certain healing promoters, such as, for example, vitamin A and synthetic inhibitors of lipid peroxidation.

A variety of antibiotics optionally can be dispersed in the polymer particles used in the invention delivery compositions to indirectly promote natural healing processes by preventing or controlling infection. Suitable antibiotics include many classes, such as aminoglycoside antibiotics or quinolones or beta-lactams, such as cefalosporins, e.g., ciprofloxacin, gentamycin, tobramycin, erythromycin, vancomycin, oxacillin, cloxacillin, methicillin, lincomycin, ampicillin, and colistin. Suitable antibiotics have been described in the literature.

Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology). In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin.

Examples of glycopeptides included in this category of antimicrobials may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc., 1996, 118, 13107-13108; J. Amer. Chem. Soc., 1997, 119, 12041-12047; and J. Amer. Chem. Soc., 1994, 116, 4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, included alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics that have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, WO 00/39156, the disclosures of which are incorporated herein by reference in their entirety.

Anti-inflammatory bioactive agents also can optionally be dispersed in polymer particles used in invention compositions and methods. Depending on the body site and disease to be treated, such anti-inflammatory bioactive agents include, e.g. analgesics (e.g., NSAIDS and salicyclates), steroids, antirheumatic agents, gastrointestinal agents, gout preparations, hormones (glucocorticoids), nasal preparations, ophthalmic preparations, otic preparations (e.g., antibiotic and steroid combinations), respiratory agents, and skin & mucous membrane agents. See, Physician's Desk Reference, 2005 Edition. Specifically, the anti-inflammatory agent can include dexamethasone, which is chemically designated as (11 [symbol] , 16I)-9-fluro-11,17,21-trihydroxy-16-methylpregna-1,4-diene-3,20-dione. Alternatively, the anti-inflammatory bioactive agent can be or include sirolimus (rapamycin), which is a triene macrolide antibiotic isolated from Streptomyces hygroscopicus.

The polypeptide bioactive agents optionally included in the invention compositions and methods can also include “peptide mimetics.” Such peptide analogs, referred to herein as “peptide mimetics” or “peptidomimetics,” are commonly used in the pharmaceutical industry with properties analogous to those of the template peptide (Fauchere, J. (1986) Adv. Bioactive agent Res., 15:29; Veber and Freidinger (1985) TINS, p. 392; and Evans et al. (1987) J. Med. Chem., 30:1229) and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂NH—, —CH₂S—, CH₂—CH₂—, —CH═CH—(cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂ NH—, CH₂CH₂—); Spatola, A. F. et al., Life Sci., (1986) 38:1243-1249 (—CH₂—S—); Harm, M. M., J. Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over natural polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a peptide (e.g., with a D-Lysine in place of L-Lysine) may be used to generate more stable peptides and peptides resistant to endogenous peptidases. Alternatively, the synthetic polypeptides covalently bound to the biodegradable polymer, can also be prepared from D-amino acids, referred to as inverso peptides. When a peptide is assembled in the opposite direction of the native peptide sequence, it is referred to as a retro peptide. In general, polypeptides prepared from D-amino acids are very stable to enzymatic hydrolysis. Many cases have been reported of preserved biological activities for retro-inverso or partial retro-inverso polypeptides (U.S. Pat. No. 6,261,569 B1 and references therein; B. Fromme et al, Endocrinology (2003) 144:3262-3269.

It is readily apparent that the subject invention can be used to prevent or treat a wide variety of diseases or symptoms thereof.

Any suitable and effective amount of the at least one macromolecular biologic and optional bioactive agent can be released with time from the polymer particles (including those in a polymer depot formed in vivo) and will typically depend, e.g., on the specific polymer, type of particle or polymer/macromolecular biologic linkage, if present. Typically, up to about 100% of the polymer particles can be released from a polymer depot formed in vivo by particles sized to avoid circulation. Specifically, up to about 90%, up to 75%, up to 50%, or up to 25% thereof can be released from the polymer depot. Factors that typically affect the release rate from the polymer are the nature and amount of the polymer, macromolecular biologic and optional bioactive agent, the types of polymer/macromolecular biologic or bioactive agent linkage, and the nature and amount of additional substances present in the formulation.

Once the invention polymer particle delivery composition is made, as above, the invention polymer compositions can be formulated for subsequent introduction to a subject by a route selected from intrapulmonary, gastroenteral, subcutaneous, intramuscular, or for introduction into the central nervous system, intraperitoneum or for intraorgan delivery. The compositions will generally include one or more “pharmaceutically acceptable excipients or vehicles” appropriate for oral, mucosal or subcutaneous delivery, such as water, saline, glycerol, polyethylene glycol, hyaluronic acid, ethanol, and the like. Additionally, auxiliary substances, such as wetting or emulsifying agents, pH buffering substances, flavorings, and the like, may be present in such vehicles.

For example, intranasal and pulmonary formulations will usually include vehicles that neither cause irritation to the nasal mucosa nor significantly disturb ciliary function. Diluents such as water, aqueous saline or other known substances can be employed with the subject invention compositions and formulations. The intrapulmonary formulations may also contain preservatives such as, but not limited to, chlorobutanol and benzalkonium chloride. A surfactant may be present to enhance absorption by the nasal mucosa.

For rectal and urethral suppositories, the vehicle used in the invention compositions and formulations will include traditional binders and carriers, such as, cocoa butter (theobroma oil) or other triglycerides, vegetable oils modified by esterification, hydrogenation and/or fractionation, glycerinated gelatin, polyalkaline glycols, mixtures of polyethylene glycols of various molecular weights and fatty acid esters of polyethylene glycol.

For vaginal delivery, the formulations of the present invention can be incorporated in pessary bases, such as those including mixtures of polyethylene triglycerides, or suspended in oils such as corn oil or sesame oil, optionally containing colloidal silica. See, e.g., Richardson et al., Int. J. Pharm. (1995) 115:9-15.

For oral delivery, molecules and vehicles with favorable physical chemical properties to reduce the solid-liquid surface tension and free energy changes and facilitate permeability across the intestinal wall, but minimal or no negative physiological/toxic properties include compounds that are Generally Recognized As Safe (GRAS), listed in the FDA Guidelines for Inactive Ingredients, or have undergone the necessary toxicity and tolerability studies as defined by official pharmaceutical regulatory agencies. Categories of molecules and vehicles that have an effect on the permeability of the intestine are bile salts, non-ionic surfactants, ionic surfactants, fatty acids, glycerides, acyl carnitines, cholines, salicylates, chelating agents, and swellable polymers. Examples of these molecules and vehicles that fall in this category include, but are not limited to natural, semisynthetic, and synthetic: phospholipids, polyethylene triglycerides, gelatin, ionic surfactants (sodium lauryl sulfate), non-ionic surfactants, e.g., dioctyl sodium sulfosuccinate, Tween® and Cremaphore®, bile acids and bile acid derivatives, digestible oils, e.g., cottonseed, corn, soybean, and olive, citric acid, EDTA, stearoyl macrogoglycerides, lauroyl macrogoglycerides, propylene glycol derivatives, i.e., propylene glycol caprylate and monocaprylate, propylene glycol laurate and monolaurate, oleoyl macrogolglycerides, caprylocaproyl macrogolglycerides, glycerol monolinoleate, glyceryl monooleate, polyglyceryl oleate, glycerol esters of fatty acids, medium chain triglycerides, sodium caprate, acyl carnitines and cholines, salicylates, e.g., sodium salicylate and methoxysalicylate, chitosan, starch, polycarbophil, N-acetylated α-amino acids, N-acetylated non-α-amino acids, 12-hydroxy stearic acid, and diethylene glycol monoethyl ether. Competitive substrates and protease inhibitors, for example compounds such as pancreatic inhibitor, soybean trypsin inhibitor, FK448, camostat mesylate, aprotinin, p-chloromericuribenzoate, and bacitracin are also included in this list.

Furthermore for oral delivery, coatings that help protect the particles from pH initiated degradation include, but are not limited to, shellac, cellulose acetate, cellulose acetate butyrate, cellulose acetate phthalate, methacrylic acid copolymers, e.g., polymethacrylate amino-ester copolymer, hydroypropyl methyl cellulose phthalate, ethyl cellulose, and poly vinyl acetate phthalate.

For a further discussion of appropriate vehicles to use for particular modes of delivery, see, e.g., Remington: The Science and Practice of Pharmacy, Mack Publishing Company, Easton, Pa., 19th edition, 1995. One of skill in the art can readily determine the proper vehicle to use for the particular macromolecular biologic/polymer particle combination, size of particle and mode of administration.

In addition to treatment of humans, the invention polymer particle delivery compositions are also intended for use in delivery of macromolecular biologics as well as bioactive agents to a variety of mammalian patients, such as pets (for example, cats, dogs, rabbits, and ferrets), farm animals (for example, swine, horses, mules, dairy and meat cattle) and race horses.

The compositions used in the invention methods will comprise an “effective amount” of the macromolecular biologic(s) of interest. For example, an amount of a macromolecular biologic will be included in the compositions for delivery thereto that will cause the subject to produce a sufficient therapeutic or palliative response in order to prevent, reduce or eliminate symptoms. The exact amount necessary will vary, depending on the subject being treated; the age and general condition of the subject to which the macromolecular biologic is to be delivered; the capacity of the subject's immune system, the degree of effect desired; the severity of the condition being treated; the particular macromolecular biologic selected and mode of administration of the composition, among other factors. An appropriate effective amount can be readily determined by one of skill in the art. Thus, an “effective amount” will fall in a relatively broad range that can be determined through routine trials. For example, for purposes of the present invention, an effective amount will typically range from about 1 μg to about 100 mg, for example from about 5 μg to about 1 mg, or about 10 μg to about 500 μg of the macromolecular biologic and, optionally, bioactive agent delivered per dose.

Once formulated, the invention polymer particle delivery compositions are administered orally, mucosally, or by subcutaneously or intramuscular injection, and the like, using standard techniques. See, e.g., Remington: The Science and Practice of Pharmacy, Mack Publishing Company, Easton, Pa., 19th edition, 1995, for mucosal delivery techniques, including intranasal, pulmonary, vaginal and rectal techniques, as well as European Publication No. 517,565 and Illum et al., J. Controlled Rel. (1994) 29:133-141, for techniques of intranasal administration.

Dosage treatment may be a single dose of the invention polymer particle delivery composition, or a multiple dose schedule as is known in the art. The dosage regimen, at least in part, will also be determined by the need of the subject and be dependent on the judgment of the practitioner. Furthermore, if prevention of disease is desired, the polymer particle delivery composition is generally administered for delivery of the macromolecular biologic prior to primary disease manifestation, or symptoms of the disease of interest. If treatment is desired, e.g., the reduction of symptoms or recurrences, the polymer particle delivery compositions are generally administered for delivery of the macromolecular biologic subsequent to primary disease manifestation.

The formulations can be tested in vivo in a number of animal models developed for the study of oral, subcutaneous, or mucosal delivery. Blood samples can be assayed for the macromolecular biologic using standard techniques, as known in the art.

The following examples are meant to illustrate, but not to limit the invention.

### EXAMPLE 1

Preparation of PEA.Ac.Bz Nanoparticles and Particles by the Single Emulsion Method

PEA polymer of structure Formula (III) containing acetylated ends and benzylated COOH groups (PEA.Ac.Bz) (25 mg) was dissolved in 1 ml of DCM and added to 5 ml of 0.1% surfactant diheptanoyl-phosphatidylcholine (DHPC) in aqueous solution while stirring. After rotary-evaporation, PEA.Ac.Bz emulsion with particle sizes ranged from 20 nm to 100 μm, was obtained. The higher the stir rate, the smaller the sizes of particles. Particle size is controlled by molecular weight of the polymer, solution concentration and equipment such as microfluidizer, ultrasound sprayer, sonicator, and mechanical or magnetic stirrer.

### EXAMPLE 2

Preparation of PEA.Ac.Bz Particles Containing a Pain Killer

PEA.Ac.Bz (25 mg) and Bupivicane (5 mg) were dissolved in 1 ml of DCM and the solution was added to 5 ml of 0.1% DHPC aqueous solution while homogenizing. Using a rotary evaporator, a PEA.Ac.Bz emulsion with average particle size ranging from 0.5 μm to 1000 μm, preferentially, from 1 μm to about 20 μm, have been made.

### EXAMPLE 3

Preparation of Polymer Particles Using a Double Emulsion Method

Particles were prepared using a double emulsion technique in two steps: in the first step, PEA.Ac.Bz (25 mg) was dissolved in 1 ml of DCM, and then 50 μl of 10% surfactant diheptanoyl-phosphatidylcholine (DHPC), was added. The mixture was vortexed at room temperature to form a Water/Oil (W/O) primary emulsion. In the second step, the primary emulsion was added slowly into a 5 ml solution of 0.5% DHPC while homogenizing the mixed solution. After 1 min of homogenization, the emulsion was rotary-evaporated to remove DCM to obtain a Water/Oil/Water double emulsion. The generated double emulsion had suspended polymer particles with sizes ranging from 0.5 μm to 1000 μm, with most about 1 μm to 10 μm. Reducing such factors as the amount of surfactant, the stir speed and the volume of water, tends to increase the size of the particles.

### EXAMPLE 4

Preparation of PEA Particles Encapsulating an Antibody Using a Double Emulsion Method

Particles were prepared using the double emulsion technique by two steps: in the first step, PEA.Ac.Bz (25 mg) was dissolved in 1 ml of DCM, and then 50 μl of aqueous solution containing 60 μg of anti-Icam-1 antibody and 4.0 mg of DHPC were added. The mixture was vortexed at room temperature to form a Water/Oil primary emulsion. In the second step, the primary emulsion was added slowly into 5 ml of 0.5% DHPC solution while homogenizing. After 1 min of homogenization, the emulsion was rotary-evaporated to remove DCM to obtain particles having a Water/Oil/Water (W/O/W) double emulsion structure. About 75% to 98% of antibody was encapsulated by using this double emulsion technique.

### EXAMPLE 5

Preparation of PEA Particles Encapsulating DNA Using a Double Emulsion Method

Particles were prepared using the double emulsion technique. In the first step, PEA.Ac.Bz (25 mg) was dissolved in 1 ml of DCM, 200 μl of DNA (0.2 mg/ml pEGFP-N1 plasmid (Clontech) in 12.5 mg/ml DHPC in water) was added, and then 50 μl of 10% surfactant diheptanoyl-phosphatidylcholine (DHPC) was added. The mixture was probe sonicated for 10 seconds to form a Water/Oil (W/O) primary emulsion. In the second step, the primary emulsion was added slowly into a 5 ml solution of 0.2% DHPC. The emulsion was vortexed and then probe sonicated for 10 seconds. The emulsion was rotary-evaporated to remove DCM to obtain a Water/Oil/Water double emulsion, which was then dialyzed in water overnight. The generated double emulsion had suspended polymer particles with sizes ranging from 0.5 μm to 1000 μm in average diameter when evaluated microscopically, with most particles about 1 μm to 10 μM in average diameter.

To determine success of DNA loading, 750 μl of particle suspension was centrifuged at 14,000×g RCF. The supernatant was harvested, and the pellet was dissolved with 700 μl ethanol to precipitate the DNA. DNA was resuspended in 50 μl water. 25 μl of each solution was placed in a 0.7% agarose gel for electrophoresis. Bands of the appropriate molecular weight for the DNA plasmid demonstrated DNA was contained in both the supernatant and the particle pellet, indicating successful, but incomplete, encapsulation.

### EXAMPLE 6

Preparation of Particles Having a Triple Emulsion Structure, Wherein One or More Primary Particles are Encapsulated Together within a Polymer Covering to Form Secondary Microparticles.

Particles having a triple emulsion structure have been prepared by the following two different routes:

Multi-particle Encapsulation. In the first route, primary particles were prepared using a standard procedure for single phase, PEA-H nanoparticles (PEA-H of formula (III) where R¹=(CH₂)₈; R²=H; R³=CH₂CH(CH₃)₂) were prepared to afford a stock sample, ranging from about 1.0 mg to about 10 mg/ml (polymer per aqueous unit). In addition, a solution of the PEA.Ac.Bz stock sample, with a 20% surfactant weight amount wherein the 20% is calculated as (milligrams of surfactant)/(milligrams of PEA.Ac.Bz+milligrams of surfactant) was prepared. Various surfactants were explored, with the most successful being 1,2-Diheptanoyl-sn-glycero-3-phosphocholine (DHPC). The stock sample of PEA-H nanoparticles was injected into a solution of PEA-AcBz polymer in DCM. A typical example was as follows:

| | |
|---|---|
| Nanoparticle Stock Solution | 100 μl |
| Dissolved PEA-AcBz | 20 mg |
| CH₂Cl₂ | 2 ml |
| Surfactant Amount | 5 mg |

This first addition was referred to as the “primary emulsion.” The sample was allowed to stir by shake plate for 5-20 minutes. Once sufficient homogeneity was observed, the primary emulsion was transferred into a canonical vial that contains 0.1% of a surface stabilizer in aqueous media (5-10 ml). These contents are referred to as the “external aqueous phase”. Using a homogenizer at low speed (5000-6000 RPM), the primary emulsion was slowly pipetted into the external aqueous phase, while undergoing low speed homogenization. After 3-5 minutes at 6000 RPM, the total sample (referred to as “the secondary emulsion”) was concentrated in vacuo, to remove the DCM, while encapsulating the PEA-Ac-H nanoparticles within a continuous PEA.Ac.Bz matrix.

Preparation of Small Molecules loaded into secondary polymer coatings. In the second route for preparing particles having a triple emulsion structure, the procedure described above for making single emulsion particles was followed for the first step. In the final step, a polymeric coating encapsulating the single emulsion particles (i.e., the water in oil phase) was then prepared.

More particularly, a water in oil phase (primary emulsion) was created. In this case, a concentrated mixture of drug (5 mg) and a surfactant (such as DHPC) was prepared first using a minimum volume of water. Then the concentrated mixture was added into a DCM solution of PEA.Ac.Bz, and was subjected to a sonication bath for 5-10 minutes. Once sufficient homogeneity was observed, the contents were added into 5 ml of water while homogenizing. After removal of DCM by vacuum evaporation, a triple emulsion of PEA.Ac.Bz containing aqueous dispersion of drug was obtained.

In another example, a stock sample of PEA-H nanoparticles with drug was prepared. PEA-H (25 mg) and drug (5 mg) were dissolved in 2 ml of DCM and mixed with 5 ml of water by sonication for 5˜10 minutes. Once sufficient homogeneity was observed, the contents were rotoevaporated to remove DCM. A typical example of preparations made using this method had the following contents.

| | |
|---|---|
| PEA-AcH | 25 mg |
| CH₂Cl₂ | 2 ml |
| H₂O | 5 ml |
| Small Molecule Drug | 5 mg |

The above preparation then was subjected to overnight evaporation in a Teflon dish to further reduce the water and yield a volume of approximately 2 ml. An exterior polymer coating, i.e. 25 mg PEA-Ac-Bz in up to 5 ml of DCM, was combined with the primary emulsion and the entire secondary emulsion was stirred by vortexing for no more than 1 minute. Finally, the secondary emulsion was transferred to an aqueous media (10-15 ml) containing 0.1% surface stabilizer, homogenized at 6000 RPM for 5 minutes, and concentrated again in vacuo to remove the second phase of DCM, thus yielding particles having a triple emulsion structure as illustrated in FIG. 6.

### EXAMPLE 7

Drug Capture (50%) by Triple Emulsion

The following example illustrates loading of a small molecule drug in a polymer coating. PEA particles containing a high loading of bupivacaine HCl were fabricated by the triple emulsion technique, using a minimal amount of H₂O in the primary emulsion, as compared to the double emulsion protocol (roughly half of the water was used). To stabilize the structure allowing for the reduction in the aqueous phase, the surface stabilizer that aides in solubilizing the drug in the aqueous droplets is dissolved itself in the internal aqueous phase before the drug is added to the internal aqueous phase. In particular, DHPC (amount below) was first dissolved into 100 μl H₂O; then 50 mg of drug was added to the phase. This technique allowed for loading of higher doses of drug in the particles, with even less water than was used in making the same sized double emulsion particles. The following parameters were followed during synthesis:

| | | | weight |
|---|---|---|---|
| Reagent | Mg | | equivalence |
| PEA-AcBz | 50 | | 50% |
| Bupivacaine HCL | 50 | | 50% |
| DHPC | 12.4 | | 20% of polymer |
| CH₂Cl₂ (solvent) | 2.5 | ml | (2% PEA in |
| | | | solvent) |
| H2O | 100 | μul | (2:1 drug) |

| | | weight |
|---|---|---|
| Reagent | Mg | equivalence |
| DHPC | 16 | 24% of polymer |
| H₂O | 5 ml | 2/1 ratio to |
| | | solvent |

### EXAMPLE 8

Process for Making Triblock Copolymer Micelles with Therapeutic Agents

First, A-B-A type triblock copolymer molecules are formed by conjugating a chain of hydrophobic PEA or PEUR polymer at the center with water soluble polymer chains containing alternating units of PEG and at least one ionizable amino acid, such as lysine or glutamate, at both ends. The triblock copolymer is then purified.

Then micelles are made using the triblock copolymer. The triblock copolymer and at least one macromolecular biologic are dissolved in aqueous solution, preferably in a saline aqueous solution whose pH has been adjusted to a value chosen in such a way that at least a portion of the ionizable amino acids in the water soluble chains is in ionized form to produce a dispersion of the triblock polymer in aqueous solution. Surface stabilizer, such as surfactant or lipid, is added to the dispersion to separate and stabilize particles to be formed. The mixed solution is then stirred with a mechanical or magnetic stirrer, or sonicator. Micelles will be formed in this way, as shown in FIG. 10, with water-soluble sections mainly on the shell, and hydrophobic sections in the core, maintaining the integrity of micellar particles. The micelles have high porosity for loading of the macromolecular biologics. Protein and other biologics can be attracted to the charged areas in the water-soluble sections. Micellar particles formed are in the size range from about 20 nm to about 200 nm.

### EXAMPLE 9

Polymer Coating on Particles Made of Different Polymer Mixed with Drug

Use of single emulsion leaves the problem that, although particles can be made very small (from 20 nm to 200 nm), the drug is matrixed in the particles and may elute too quickly. For double and triple emulsion particles, the particles are larger than is prepared by the single emulsion technique due to the aqueous solution inside. However, if the same polymer is used for coating the particles as is used to matrix the drug, the solvent used in making the third emulsion (the polymer coating) will dissolve the matrixed particles, and the coating will become part of the matrix (with drugs in it). To solve this problem, a different polymer than is used to matrix the drug is used to make the coating of the particles and the solvent used in making the polymer coating is selected to be one in which the matrix polymer will not dissolve.

For example, PEA can be dissolved in ethanol but PLA cannot. Therefore, PEA can be used to matrix the drug and PLA can be used as the coating polymer, or vice versa. In another example, ethanol can dissolve PEA but not PEUR and acetone can dissolve PEUR but cannot dissolve PEA. Therefore, PEUR can be used to matrix the drug and PEA can be used as the coating polymer, or vice versa.

Therefore, the general process to be used is as follows. Using polymer A, prepare particles in solution (aqueous if polymer A is PEA, PEUR of PEU) using a single emulsion procedure to matrix drug or other bioactive agent in the polymer particles. Dry out the solvent by lyophilization to obtain dry particles. Disperse the dry particles into a solution of polymer B in a solvent that does not dissolve the polymer A particles. Emulsify the mixture in aqueous solution. The resulting particles will be nanoparticles with a coating of polymer B on particles of polymer A, which contain matrixed drug.

### EXAMPLE 10

Preparation of Insulin-Polymer Conjugate Using an Activated Ester Method

Materials. N,N-diisopropylethylamine (DIPEA), 1-ethyl-(3-dimethylaminopropyl) carbodiimide hydrochloride (EDC), N-hydroxysuccinimide (HOSu), diisopropylethylamine (DIPEA), n-hydroxysuccinimide (HNS), dichloromethane (DCM), dioleoyl phosphotidylchloline (DOPC), Dimethylsufloxide (DMSO), 1,1,1,3,3,3-hexafluoro isopropanol (HFIP), trifluoroethanol (TFE), polyvinyl alcohol (PVA), polyvinylpyrrolidone (PVP), N,N-dimethylformamide (DMF), acetonitrile (ACN) were purchased from Aldrich Chemical CO., Milwaukee, Wis. and used without further purification. Other solvents, acetone, hexanes, and ethanol (Fish

This Example illustrates covalent attachment of insulin to PEA polymer via amino groups therein. Because insulin has multiple attachment sites (i.e. three primary amino groups per molecule), conjugation to the polymer can be either at a single-site, (where the insulin molecule is attached to only one carboxyl of PEA polymer) or at multiple sites, (where more than one carboxylate, either of a polymer chain or from polymer chains, is bound per one molecule of insulin). The case of attachment at multiple sites can be detected by various techniques. For example, the changes in average molecular weight and weight distribution can be monitored by GPC.

PEA-H of formula (III) where R¹=(CH₂)₈; R²=H; R³=CH₂CH(CH₃)₂, with the molecular weight of 59,000 g/mol and a polydispersity of 1.557, was first activated in DMF using N-hydroxysuccinimide (NHS) and DCC as conjugating agent. This involved dissolving of 0.607 g of PEA-H (328 μmol) in 2.8 mL of DMF under argon and then stirring the clear reaction mixture in the presence of 37.3 mg of DCC (181 μmol, ca. 0.55 equivalents) and 20.8 mg of NHS (181 μmol, 0.55 equivalents) at room temperature for approximately 24 hours. The reaction mixture was then filtered through a 0.45 μm pore sized frit, which was then rinsed with 1 mL of DMF. The resultant PEA-OSu solution was further conjugated without isolation.

The N-hydroxysuccinamide activated PEA, designated as PEA-OSu_{z}, (where z ranges from 0 to p and R² is succinimide residue) was further reacted with insulin. The conjugation of insulin to the activated polymer was accomplished by adding a pre-determined amount of insulin solution in DMSO. More particularly, insulin conjugation to the polymer was carried out as follows: 0.990 g of insulin (165 μmol, 0.9 equivalents) was dissolved separately in 6.7 mL of DMSO. The insulin solution and 86 μL DIPEA (497 μmol, 3.0 equivalents) was added to the activated PEA-OSu solution and stirred for 48 hours. Total concentration of insulin in the reaction mixture was 86.8 mg/mL. The reaction solution was either forwarded to insulin-hexamer processing or precipitated in 15 mL ether/acetone (1:1) and collected by centrifuge at 3600 rpm at 4° C. for 15 min. In order to remove the residual free insulin, the PEA-Insulin conjugates were washed several times in pH=3.7 buffer. The residual free insulin peak was monitored by GPC.

The PEA-Insulin conjugate was analyzed by GPC and had a molecular weight of 204,000 g/mol and a polydispersity of 2.28 as summarized in Table 3. The molecular weight of the sample exceeded the maximum molecular weight expected for single-site attachment, which indicated that cross-linking had occurred.

In order to better control the cross-linking, the PEA-Insulin conjugate reactions were performed in various dilute concentrations in DMSO (6×, 10×, 17×, refer to above reaction solution 86.8 mg/mL). As displayed in Table 3, the molecular weights and polydispersities of the diluted reactions were significantly lower than the previous reaction and within the expected range for single-site attachment. This result signifies that intermolecular crosslinking was no longer occurring and that the intra-chain linked product was achieved.

TABLE 3
Molecular weights of PEA-Insulin conjugates achieved after
applying insulin solutions in various concentrations

| | | Insulin | | | |
|---|---|---|---|---|---|
| | | concentration | | | |
| | PEA -Conjugate^{a)} | in solution | Mw^{b)} | Mn^{b)} | |
| # | (×insulin dilution) | [mg/mL] | [Da] | [Da] | Mw/Mn^{b)} |
| 1 | PEA-H | — | 58800 | 37800 | 1.56 |
| 2 | PEA-Insulin (×1) | 86.8 | 204000 | 89000 | 2.28 |
| 3 | PEA-Insulin (×6) | 17.4 | 106000 | 54000 | 1.96 |
| 4 | PEA-Insulin (×10) | 8.85 | 96300 | 52600 | 1.83 |
| 5 | PEA-Insulin (×17) | 5.10 | 82700 | 48900 | 1.69 |

^{a)}For each experiment 0.607 g of PEA-H (328 μmol) was conjugated.
^{b)}GPC Measurements were carried out in DMAc, (PS)

Insulin-hexamer formation and crystallization The polymer-insulin conjugate, PEA-Insulin_{z}, was dissolved in DMSO, diluted 1:4 volume ratio with a buffer containing zinc sulfate and phenol at pH 6.5, and then added to a dialysis tube with a molecular weight cutoff of 3000 g/mol. Then an additional 5 equivalents of insulin was added to the dialysis bag for every equivalent of insulin covalently attached to the polymer. The contents of the dialysis bag were stirred for three to four days in a crystallization buffer of zinc sulfate, phenol, pH 6.5, with the crystallization buffer being changed three times every day. The solid in the dialysis bag was then lyophilized and analyzed by gel permeation chromatography for percent (w/w) of insulin loading per polymer-insulin conjugate (PIC).

### EXAMPLE 11

Preparation of Ovalbumin-Polymer Conjugate Using Activated Solvated Ester Method

Conjugation of ovalbumin (OVA) to the activated polymer, PEA-OSu_{z}, was accomplished by dissolving a predetermined volume of OVA to DMSO, with an equivalent volume of DIPEA, in a reaction flask containing the activated polymer prepared as in Example 10 to produce the polymer-OVA conjugate, PEA-OVA_{z}. The reaction was conducted under argon for 72-hrs at room temperature. The reaction solution was then extracted with 3×2-mL ether by centrifuging for 15-min at 3600 rpm at 4° C. and the remaining ether was removed. The white pellet obtained was then extracted with 3×15-mL water by centrifuging for 15 min at 3600 rpm at 4° C. The OVA-polymer conjugate, PEA —OVA_{z}, was then dried on the lyophilizer.

### EXAMPLE 12

Preparation of a Polymer Matrix Containing Insulin as Macromolecular Biologic

Method 1: The polymer PEA-H of structural Formula (III) wherein R¹=(CH₂)₈; R²=H; R³=CH₂CH(CH₃)₂, and free insulin were dissolved in HFIP/Dioxane (1:10 v/v) with a different coating polymer (i.e., PEA of formulas (I) and (III), PEUR of formulas (IV) and (V), or PEU of formulas (VI) and (VII)) in a 1:2 volume ratio and the solution was stirred until both polymers were completely dissolved. This solution was then mixed 1:10 volume ratio in dioxane, frozen, and lyophilized to obtain an amorphous material having the polymer PEA-Insulin_{z} conjugate matrixed in PEUR formula (V), wherein R⁶=(CH₂)₈; R²=H; R³=CH₂CH(CH₃)₂, R⁴=DAS (of structural Formula II), wherein m=3, p=1; 120 KDa.

Method 2: The PEA-H and free insulin were dissolved in HFIP overnight and then another coating polymer (i.e., PEA of formulas (I) and (III), PEUR of formulas (IV) and (V)) in HFIP was added in a 2:1 volume ratio. The solution was stirred until both polymers were completely dissolved. This solution was then mixed 1:10 volume ratio in dioxane, frozen, and lyophilized to obtain an amorphous material having the polymer-insulin conjugate, PEA-Insulin_{z} matrixed in coating polymer PEUR.Ac.Bz. of formula (V), where R²=—CH₂C₆H₅, R³=—CH₂CH(CH₃)₂, R⁴=DAS, R⁶=(CH₂)₃, m=3, p=1.

### EXAMPLE 13

Preparation of Nanospheres Containing Polymer-Encapsulated Insulin

Recombinant human insulin in large particles was completely dissolved into acetic acid and the solution formed was placed into dialysis tubing and dialyzed against DCM until a precipitate was formed (the time can vary from 1-48 hrs and the temperatures can vary from 5-50° C.) without agitation. Surfactants (PVA, PVP, dextrin etc.) can be added to the insulin solution prior to dialysis if necessary. The precipitate in the form of nanoparticles of insulin was collected and lyophilized to obtain a white powder.

Various ratios of coating polymer PEA. H and polymer-insulin conjugate, PEA-Insulin_{z}, and 20 mg of DOPC were dissolved in DCM to obtain a polymer solution having a polymer concentration of 100 mg/ml. Then 10 mg of the insulin nanoparticles dispersed in DCM were mixed with the polymer solution by vortexing to give a 20 ml solution. This solution was added to 25˜100 ml of aqueous phase containing 5˜50 mg of SLS (additional surfactants like PVA can be added to the aqueous phase in a polymer/surfactant ratio from 1 to 5). The resulting mixture was shaken, vortexed and mixed by ultra-sonication for 5˜100 seconds to form a water/oil emulsion, which was then roto-evaporated to remove all of the residue organic solvent to stabilize the product nanoparticles. The insulin encapsulated in polymer nanoparticles can then be stored in solution or further lyophilized to obtain white powders. The lyophilized nanoparticles obtained can be re-dispersed in aqueous solution at room temperature.

### EXAMPLE 14

Preparation of Free Ovalbumin Encapsulated in Polymer Microspheres Using the Oil Organic in Polar Organic (o/o) Emulsion Technique.

20 mg. of PEA-H and a predetermined amount (4-5 mg) of ovalbumin were dissolved in about. 3 ml of HFIP. The coating polymer PEUR.Ac.Bz (polymer of formula (V) where R²=H, R³=—CH₂CH(CH₃)₂, R⁴=DAS, R⁶=(CH₂)₃, m=3, p=1), was dissolved in 3 mL HFIP and the two solutions were added together to obtain microspheres by the oil-in-oil (o/o) dispersion method (Murty et al. AAPS PharmSciTech. 2003; 4:E50, Bodmeier and Hermann, Eur. J. Pharm Biopharm. (1998), p 75-82). The mixture of polymers was then emulsified for 30 minutes (at 6000 rpm, 40° C.) in 80-ml cottonseed oil containing 0.4 ml of a stabilizer, sorbitan monooleate to produce microspheres encapsulating the ovalbumin. The HFIP was removed by roto-evaporation from the solution containing the microspheres. The resulting solution was then diluted with a three fold volume of hexane and the microspheres were collected by vacuum filtration through a PTFE 0.45 micron filter. The microspheres were removed from the filter and dried by lyophilization.

### EXAMPLE 15

Preparation of an Amorphous Material in which Insulin is Protected in a Polymer Matrix:

Method 1. The PEA-Insulin_{z}, conjugate was dissolved in DCM with a different coating polymer (for example, PEA of formula (I) and (III), PEUR of formula (IV) and (V), or PEU of formula (VI) and (VII) can be used) in a 1:2 volume ratio. The solution was stirred until both polymers were completely dissolved. Then this solution was dissolved 1:10 volume ratio in dioxane and lyophilized to obtain an amorphous material in which the conjugate PEA-Insulin_{z} is matrixed in PEA of Formula (III), where, R¹ is a equimolar mixture of (CH₂)₈ and CPP, R²=—CH₂C₆H₅, R³=—CH₂CH(CH₃)₂, R⁴=(CH₂)₆, m=3, p=1.

Method 2. The PEA-Insulin_{z} conjugate was dissolved in HFIP/Dioxane (1:10 volume ratio) with a different coating polymer (i.e. PEA, PEUR, PEU etc.) in a 1:2 volume ratio. The solution was stirred until both polymers were completely dissolved. Then this solution was frozen in liquid nitrogen and lyophilized to obtain an amorphous material in which conjugate PEA-Insulin_{z} is matrixed in PEUR of formula (V), where, R²=—CH₂C₆H₅, R³=—CH₂CH(CH₃)₂, R⁴=DAS, R⁶=(CH₂)₃, m=3, p=1

Method 3. The PEA-Insulin_{z} conjugate was dissolved in HFIP overnight and then a different coating polymer (i.e. PEA, PEUR, PEU etc.) in HFIP was added in a 2:1 volume ratio. The solution was mixed and frozen in liquid nitrogen and lyophilized to obtain an amorphous material wherein the conjugate PEA-Insulin_{z} is matrixed in PEUR of formula (V), where, R²=—CH₂C₆H₅, R³=—CH₂CH(CH₃)₂, R⁴=DAS, R⁶=(CH₂)₃, m=3, p=1.

### EXAMPLE 16

Preparation of Polymer Coated Insulin-Containing Nanospheres by w/o Emulsion Technique

Approximately 100 mg of an encapsulating polymer (PEA, PEUR etc.) and 20 mg of DOPC were co-dissolved in DCM to obtain a polymer solution with a polymer/DOPC concentration of 100 mg/ml. Then 10 mg of PEA-Insulin_{z} conjugate dispersed in DCM was mixed with the polymer solution by vortexing to give a 20 ml solution. To this solution was added 25˜100 ml of aqueous phase containing 5˜50 mg of SLS (additional surfactants like PVA can be added to the aqueous phase in a PVA to polymer ratio from 1 to 5). This mixture was shaken, vortexed and mixed by ultra-sonication for 5˜100 seconds, then roto-evaporated to remove all of the residue organic solvent to stabilize the nanoparticles. The insulin nanoparticles can then be stored in solution or further lyophilized to obtain white powders. The powder of polymer coated insulin nanoparticles can be re-dispersed in aqueous solution at room temperature.

### EXAMPLE 17

Preparation of Polymer Coated Insulin-Containing Nanospheres by o/o Emulsion Technique

The PEA-Insulin_{z} conjugate and PEUR polymer of formula (V), where, R²=—CH₂C₆H₅, R³=—CH₂CH(CH₃)₂, R⁴=DAS, R⁶=(CH₂)₃, m=3, p=1, were dissolved completely in 6 mL of HFIP. This solution was added slowly through a 27-gauge stainless steel needle to a rapidly stirring solution (6000 rpm) of 80-mL of cottonseed oil and 0.4 ml of sorbitan monooleate at 40° C. for 10 min to obtain microspheres by the oil-in-oil (o/o) dispersion method (Murty et al., supra and Bodmeier and Hermann, supra). The HFIP/TFE was then removed by roto-evaporation for 40 min in a water bath at a temperature of 40° C. The resulting microspheres in solution were obtained by diluting the solution with three times more hexane and filtering this solution through a 0.45 micron PTFE filter. The product microspheres were removed from the surface of the filter and lyophilized overnight to obtain a fine white powder.

### EXAMPLE 18

Encapsulation of Ovalbumin —Polymer Conjugate in Microspheres Using Oil Organic in Polar Organic (o/o) Emulsion Technique

Preparation of polymer coated ovalbumin-conjugate (I) The PEA —OVA_{z} conjugate and PEUR polymer of formula (V), where, R²=—CH₂C₆H₅, R³=—CH₂CH(CH₃)₂, R⁴=DAS, R⁶=(CH₂)₃, m=3, p=1, were dissolved completely in 6 mL of HFIP. This solution was added slowly through a 27-gauge stainless steel needle to a rapidly stirring solution (6000 rpm) of 80 mL of cottonseed oil and 0.4 ml of sorbitan monooleate at 40° C. for 10 min to obtain microspheres by the oil-in-oil (o/o) dispersion method (Murty et al., supra and Bodmeier and Hermann, supra). The HFIP/TFE was then removed by roto-evaporation for 40 min in a water bath with a temperature of 40° C. The resulting microspheres in solution were obtained by diluting the solution with three-fold volume of hexane and filtering this solution through a 0.45 micron PTFE filter. The microspheres were removed from the surface of the filter and lyophilized overnight to obtain a fine white powder.

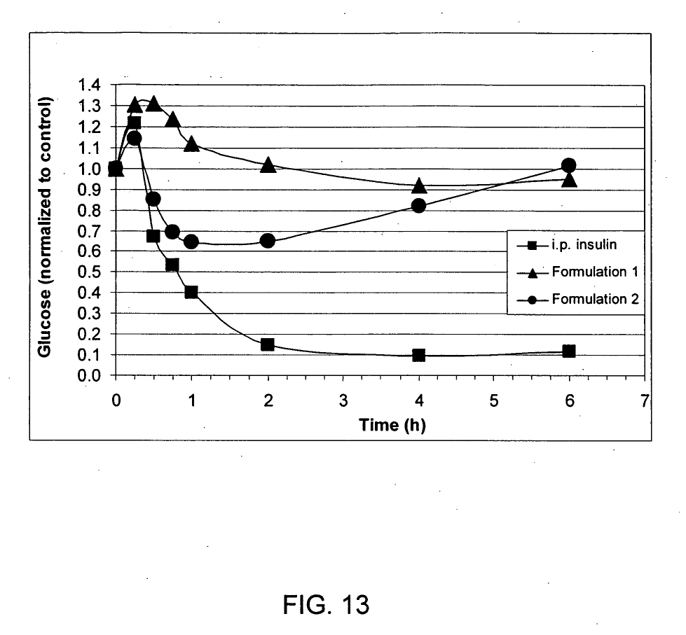

Preparation of Polymer Coated Insulin-Conjugates (II) (see Table 4, FIG. 12)

Method 1. Insulin (11.55 mg), DOPC (40 mg), and PEA.Ac.Bz (100 mg) was dissolved in 6 ml of DCM. This mixture was vortexed, sonicated and rotoevaporated after being added to a 0.25% DHPC (0.25%) aqueous solution. This solution was reduced to 8 mL.

Method 2. Added 60 mg of PEA-Ins conjugate and dissolved in 8.0 ml DCM. Added 30 mg of PEUR (85 kDa) dissolved in 4.0 ml of DCM. Added the polymer solution to the PEA construct and mix them together to obtain a turbid solution. Added 6.0 mL of hexanes to the polymer solution. The solution became cloudy. Then added 18 mL of dioxane and the solution became clear. The material was lyophilized to obtain a white amorphous powder.

TABLE 4
Formulations exemplifying polymer coated insulin-conjugate (II)

| Formulation | Insulin | Coating Polymer |
|---|---|---|
| 1,16-1r4 | Insulin | MVPEA.I.Ac.Bz |
| 2,16-1 | Insulin | MVPEA.I.Ac.Bz |
| 3 | PEA(65kDa)[Ins- | PEA(41kDa).8- |
| | Hex].Ac | CPP(50%)Ac.Bz |
| 4 | PEA(65kDa)[Ins- | PEUR(85kDa)-8- |
| | Hex].Ac | Phe(DA).Ac.H. |

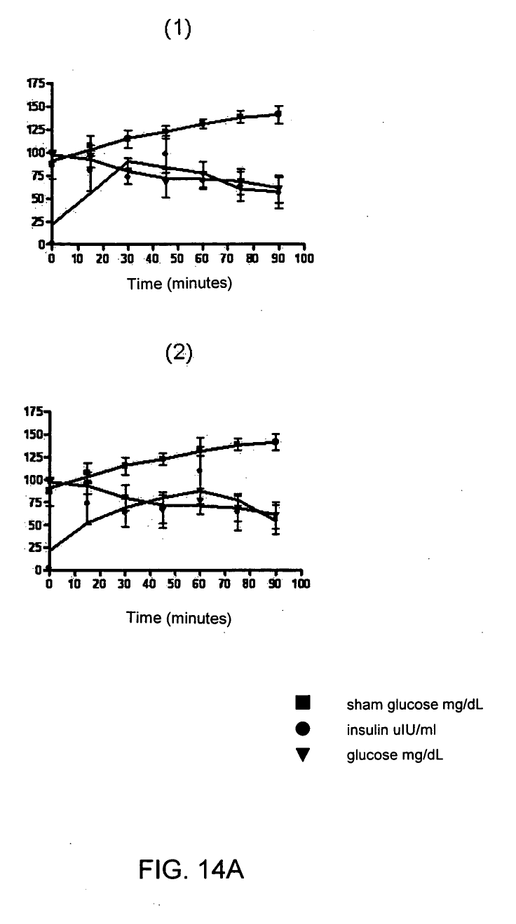

Preparation of Polymer Coated Insulin-Conjugates (III) (see Table 5, and FIG. 13).

Method 1. The PEA (65 kDa)[Ins-HEX] (150 mg) was dissolved in 3 ml of DCM and mixed with 75 mg PEA (41 kDa)-8-CPP (50%).Ac.Bz in 1.5 ml of DCM. The 15 mL of dioxane was added to the mixture and the solution was lyophilized. This product was called formulation 1.

Method 2. The insulin (35 mg), DOPC (63 mg) and PEA.Ac.Bc./PEA-H (8:2) (315 mg) were dissolved in 38.7 mL of DCM. This mixture was then vortexed, sonicated, and emulsified in 100 ml 0.05% PVA (80) The solution was roto-evaporated and lyophilized overnight.

TABLE 5
Formulations exemplifying polymer coated insulin-conjugate (III)

| Formu- | | |
|---|---|---|
| lation | Insulin | Polymer 1 |
| 1 | PEA(65kDa)[Ins-Hex].Ac- | PEA (41 kDa)-8-CPP(50%).Ac.Bz |
| | insulin polymer conjugate | |
| 2 | Human Insulin | PEA.Ac.Bz./ PEA-H (8:2) |
| | (non-conjugate) | |

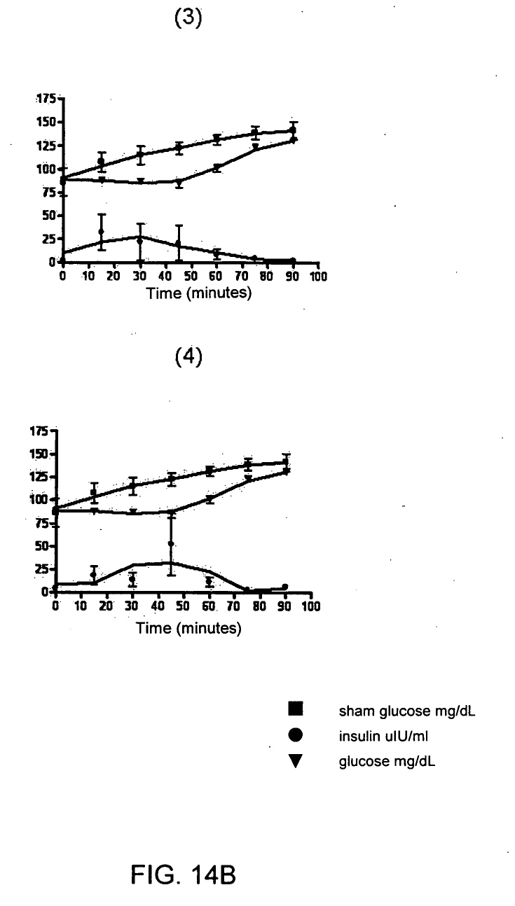

Preparation of Polymer Coated Insulin Conjugates (IV) (see Table 6, and FIGS. 14a-14b

Method 1—Samples. The following materials were used to make the oral insulin formulations in different combinations: PEA (65 kDa).H.Ac, PEA-4PheDasAcBz, PEA[Ins]₆, Oleic acid, triglycerides, Span 80, palmitoyl carnatine, and PVA. The oral insulin microspheres were made according the to the oil-in-water single emulsion method as described previously. The individual ingredients of the formulations are given in table 6.

TABLE 6
Formulation exemplifying polymer coated insulin-conjugate (IV)

| | | | PEA(83 kDa)-4- | | SPAN | |
|---|---|---|---|---|---|---|
| Formulation | PEA[Ins]₆ | PEA(65 kDA)•H•Ac | Phe(DAS)•Ac•Bz | Triglycerides | 80 | PVA |
| 154-W | 80 | 45 | 75 | 90 | 15 | 30 |

Triglycerides = 1:1 capric:caprylic triglycerides;
SPAN 80 = Polysorbate Monooleate;
PVA = polyvinyl alcohol (80% hydrolyzed).

### EXAMPLE 19

Recovery of Biologically Active Insulin from Particles

Particles containing insulin were prepared using either a double emulsion technique or by seeding of oligomerization and crystallization of the insulin by the technique using polymer-biologic conjugates. Particles were centrifuged and dissolved with DCM to recover the insulin. L6 rat skeletal muscle cells were grown to confluence in 60 mm dishes in 10% FBS/90% DMEM (Cambrex) and then the medium was changed to 2% FBS/98% DMEM to increase the efficiency of differentiation from myoblasts to myotubes for assay. On the day of assay, the cells were depleted of serum for 2 hours, then rinsed with PBS. The insulin (normalized from all samples to 100 nM) was then applied to L6 cell cultures to measure biological activity of insulin through its ability to stimulate AKT phosphorylation. Following a 5 minute exposure of the cells to the insulin at room temperature with rocking, the cell culture plates were placed on ice and rinsed with PBS containing 1 mM sodium orthovanadate. The cells were scraped from the surface of plates using a cell scraper, pipetted into a 1.5 ml Eppendorf tubes, and centrifuged to pellet the cells. 40 μl of lysis buffer was added to each tube and incubated with cells for 15 minutes on ice. Lysates, were centrifuged to remove debris and then assayed for the degree of AKT phosphorylation using standard Western blotting techniques. Bands of the appropriate molecular weight (65 kDa) were detected in the lanes on the blot that had been loaded with insulin from the particle formulations. By this method, it was demonstrated that the insulin incorporated in the particle formulations retained its functional ability to stimulate cell signaling, as measured by phosphorylation of AKT.

### EXAMPLE 20

Delivery of Biologically Active Insulin from Particles Decreases Systemic Glucose Levels in Hyperglycemic Mice and Rats

Particles containing insulin were prepared according to methods II-IV in Example 18. The formulations were delivered by oral gavage to hyperglycemic mice or rats. Fasting blood glucose (FBG) was measured from peripheral blood samples following treatment with insulin. Decreases in FBG, as shown by the results summarized in FIGS. 12 (Example 18, method II) and 13 (Example 18, method III), demonstrate that biologically active insulin was released from the particles and effected a change in the glucose levels in the blood. No change in FBG is a value of 1.0 on the graphs. In the mouse trial (FIG. 12), 10-50% reductions in FBG were achieved over about 2 hours. In the rat trial (FIG. 13), a 35% reduction in FBG was achieved over about 3 hours. The reduction achieved by the particles was about 29% as effective in reducing FBG as the positive control, intraperitoneal (i.p.) injection of insulin, as measured by areas over the curve in the graphs shown in FIGS. 12 and 13.

### EXAMPLE 21

Delivery of Biologically Active Insulin from Particles Decreases Systemic Glucose Levels and Delivers Insulin into the Bloodstream of Normoglycemic Rats (Preclinomics Study).

To understand the mechanism involved in oral insulin delivery, a study was devised to examine the ability of the PEA-Insulin conjugate particles to deliver insulin from the duodenum into the portal and peripheral circulatory system. PEA-Insulin conjugate particles were fabricated by seeding, oligomerization and crystallization of the insulin by the technique using polymer-biologic conjugates as described above in Example 18, method IV.

Male Sprague Dawley rats were fasted overnight and placed under anesthesia the next morning so that catheters (Becton Dickinson Saf-T-Intima™ Winged IV Cath System, 22G×¾″) could be placed into the duodenum and the portal vein. Following catheter placement, the incision was closed leaving external access via the catheter tubing.

The rats were placed on warming pads to maintain proper body temperature throughout the experiment. The test particles were injected into the duodenal catheter and human insulin was delivered SubQ. Blood samples were taken from the portal catheter and from the tail vein to determine glucose and insulin concentrations in the portal and peripheral circulation. Due to the technical nature of the surgery, not all rats survived, resulting in varying numbers of rats for each test group; however, all the PEA-Insulin groups had a minimum of five rats (n=5).

Blood samples were taken at 0, 15, 30, 45, 60, 75 and 90 minutes post dosing. Glucose analysis was done with a One Touch Glucometer using freshly drawn blood. Insulin samples were allowed to clot and then spun to isolate plasma. Human insulin samples were assayed using the Mercodia Ultrasensitive Insulin ELISA (ALPCO).

The graphs in FIG. 15A, Panel A, show the averaged human insulin and rat glucose data for groups 1, 2 and 6. The graphs in FIG. 15B show the averaged human insulin and rat glucose data for groups 3, 4 and 5. The top 3 graphs in each of FIGS. 15A and B represent samples taken from the portal circulation, and the bottom 3 graphs in each of FIGS. 15A and B show data from the peripheral circulation. In addition the glucose levels taken from sham animals (which underwent surgery but did not receive any test particles) are used as a control to demonstrate the glucose profile for rats in the absence of any human insulin. In both panels, the presence of human insulin above the background of endogenous rat insulin results in a lowering of glucose levels.

The catheterized rat studies clearly demonstrate the ability of the PEA-Insulin conjugate particles to deliver human insulin from the duodenum to the portal and peripheral circulation. The presence of this exogenous insulin results in a lowering of the rat glucose levels when insulin is delivered rapidly and in a sufficient quantity.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

Although the invention has been described with reference to the above examples, it will be understood that modifications and variations are encompassed within the spirit and scope of the invention. Accordingly, the invention is limited only by the following claims.

What is claimed is:

1. A polymer particle delivery composition comprising at least one macromolecular biologic conjugated via at least one site thereof to a biodegradable polymer so as to maintain the native activity of the macromolecular biologic, wherein the polymer comprises at least one or a blend of the following:
a poly(ester amide) (PEA) having a chemical formula described by structural formula (I), 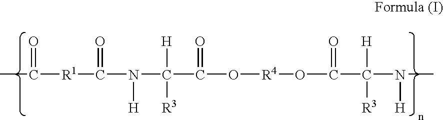
wherein n ranges from about 5 to about 150; R¹ is independently selected from residues of α,ω-bis (o, m, or p-carboxyphenoxy)-(C₁-C₈) alkane, 3,3′-(alkanedioyldioxy)dicinnamic acid or 4,4′-(alkanedioyldioxy)dicinnamic acid, (C₂-C₂₀) alkylene, and (C₂-C₂₀) alkenylene; the R³s in individual n monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; and R⁴ is independently selected from the group consisting of (C₂-C₂₀) alkylene, (C₂-C₂₀) alkenylene, (C₂-C₈) alkyloxy, (C₂-C₂₀) alkylene, saturated or unsaturated therapeutic diol residues, or bicyclic-fragments of 1,4:3,6-dianhydrohexitols of structural formula (II), and combinations thereof; 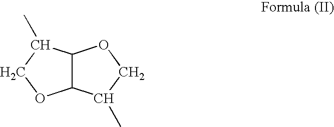
or a PEA polymer having a chemical formula described by structural formula (III): 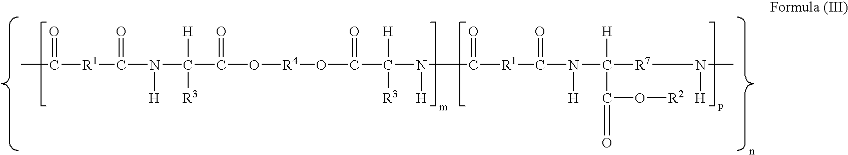
wherein n ranges from about 5 to about 150, m ranges about 0.1 to 0.9: p ranges from about 0.9 to 0.1; wherein R¹ is independently selected from residues of α,ω-bis (o, m, or p-carboxyphenoxy) (C₁-C₈) alkane, 3,3′-(alkanedioyldioxy)dicinnamic acid or 4,4′-(alkanedioyldioxy) dicinnamic acid, (C₂-C₂₀) alkylene, or (C₂-C₂₀) alkenylene; the R³s in individual m monomers are independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; R⁴ is independently selected from the group consisting of (C₂-C₂₀) alkylene, (C₂-C₂₀) alkenylene, (C₂-C₈) alkyloxy, (C₂-C₂₀) alkylene, saturated or unsaturated therapeutic diol residues, bicyclic-fragments of 1,4:3,6-dianhydrohexitols of structural formula (II), and combinations thereof; and R⁷ is independently (C₁-C₂₀) alkyl or (C₂-C₂₀) alkenyl;
or a (ester urethane) (PEUR) having a chemical formula described by structural formula (IV), 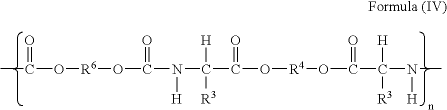
wherein n ranges from about 5 to about 150; wherein R³s in independently selected from the group consisting of hydrogen, (C₁-C₆) alkyl, (C₂-C₆) alkenyl, (C₂-C₆) alkynyl, (C₆-C₁₀) aryl (C₁-C₂₀) alkyl, —(CH₂)₂SCH₃; R⁴ is selected from the group consisting of (C₂-C₂₀) alkylene, (C₂-C₂₀) alkenylene or alkyloxy, saturated or unsaturated therapeutic diol residues and bicyclic-fragments of 1,4:3,6-dianhydrohexitols of structural formula (II); and R⁶ is independently selected from (C₂-C₂₀) alkylene, (C₂-C₂₀) alkenylene or alkyloxy, bicyclic-fragments of 1,4:3,6-dianhydrohexitols of general formula (II), a residue of a saturated or unsaturated therapeutic diol, and combinations thereof;
or a PEUR polymer having a chemical structure described by general structural formula (V): 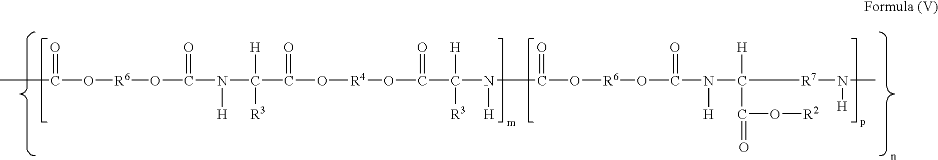
wherein n ranges from about 5 to about 150, m ranges about 0.1 to about 0.9: p ranges from about 0.9 to about 0.1; R² is independently selected from hydrogen, (C₆-C₁₀) aryl (C₁-C₂₀) alkyl, or a protecting group; the R³s in an individual m monomer are independently selected from the group consisting of hydrogen, (C₁-C₆) alkyl, (C₂-C₆) alkenyl, (C₂-C₆) alkynyl, (C₆-C₁₀) aryl(C₁-C₂₀) alkyl, and —(CH₂)₂SCH₃; R⁴ is selected from the group consisting of (C₂-C₂₀) alkylene, (C₂-C₂₀) alkenylene or alkyloxy, a residue of a saturated or unsaturated therapeutic diol and bicyclic-fragments of 1,4:3,6-dianhydrohexitols of structural formula (II); R⁶ is independently selected from (C₂-C₂₀) alkylene, (C₂-C₂₀) alkenylene or alkyloxy, bicyclic-fragments of 1,4:3,6-dianhydrohexitols of general formula (II), an effective amount of a residue of a saturated or unsaturated therapeutic diol, and combinations thereof; and R⁷ is independently (C₁-C₂₀) alkyl or (C₂-C₂₀) alkenyl;
or a poly(ester urea) (PEU) having a chemical formula described by general structural formula (VI): 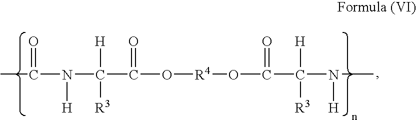
wherein n is about 10 to about 150; the R³s within an individual n monomer are independently selected from hydrogen, (C₁-C₆) alkyl, (C₂-C₆) alkenyl, (C₂-C₆) alkynyl, (C₆-C₁₀) aryl (C₁-C₂₀)alkyl, and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, an effective amount of a residue of a saturated or unsaturated therapeutic diol; or a bicyclic-fragment of a 1,4:3,6-dianhydrohexitol of structural formula (II), and combinations thereof;
or a PEU having a chemical formula described by structural formula (VII) 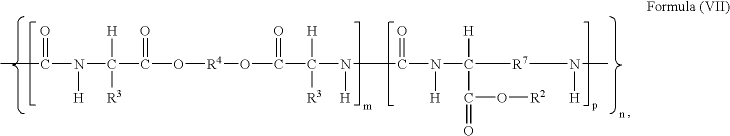
wherein m is about 0.1 to about 1.0; p is about 0.9 to about 0.1; n is about 10 to about 150; R² is independently hydrogen, (C₁-C₁₂) alkyl or (C₆-C₁₀) aryl or a protective group; the R³s within an individual m monomer are independently selected from hydrogen, (C₁-C₆) alkyl, (C₂-C₆) alkenyl, (C₂-C₆) alkynyl, (C₆-C₁₀) aryl (C₁-C₂₀) alkyl, and —(CH₂)₂SCH₃; R⁴ is independently selected from (C₂-C₂₀) alkylene, (C₂-C₂₀) alkenylene, (C₂-C₈) alkyloxy (C₂-C₂₀) alkylene, a residue of a saturated or unsaturated therapeutic diol; or a bicyclic-fragment of a 1,4:3,6-dianhydrohexitol of structural formula (II), and combinations thereof; and R⁷ is independently (C₁-C₂₀) alkyl or (C₂-C₂₀) alkenyl.

2. The composition of claim 1, wherein the macromolecular biologic is in the form of a protein, polypeptide, oligopeptide, peptide, polynucleotide, oligonucleotide, or nucleic acid.

3. The composition of claim 2, wherein at least one of the macromolecular biologics is conjugated to the polymer via more than one site thereon to cross-link the polymer.

4. The composition of claim 2, wherein the macromolecular biologic is in the form of an oligomer.

5. The composition of claim 4, wherein the oligomer is an insulin oligomer

6. The composition of claim 5, wherein the insulin oligomer is a sextet of insulin promoters.

7. The composition of claim 1, wherein the macromolecular biologic is in the form of a protein crystal or aggregate.

8. The composition of claim 7, wherein the protein crystal or aggregate further comprises at least one atom of calcium or a transition metal.

9. The composition of claim 7, wherein the protein aggregate is a crystal of insulin oligomers.

10. The composition of claim 9, wherein the crystal of insulin oligomers further comprises at least one zinc atom.

11. The composition of claim 1, wherein the composition is formulated for oral delivery.

12. The composition of claim 11, wherein the composition further comprises at least one bile salt matrixed in the polymer that is natural for the species of the subject to which the composition is intended for delivery

13. The composition of claim 12, wherein the species of the subject is human and the bile salt is based on cholic acid.

14. The composition of claim 4, wherein the oligomer is of a therapeutic protein.

15. The composition of claim 8, wherein the crystal or aggregate is of a therapeutic protein.

16. The composition of claim 1, wherein the polymer comprises a PEA described by structural formula (III) or (IV).

17. The composition of claim 16, wherein at least one R¹ is a residue of α,ω-bis (o, m, or p-carboxyphenoxy) (C₁-C₈) alkane, 3,3′-(alkanedioyldioxy)dicinnamic acid, or 4,4′(alkanedioyldioxy)dicinnamic acid, or at least one R⁴ is a bicyclic-fragment of a 1,4:3,6-dianhydrohexitol of structural formula (II).

18. The composition of claim 16, wherein at least one R¹ is a residue of α,ω-bis (o, m, or p-carboxyphenoxy) (C₁-C₈) alkane, 3,3′-(alkanedioyldioxy)dicinnamic acid, or 4,4′-(alkanedioyldioxy)dicinnamic acid, or a mixture thereof, and at least one R⁴ is a bicyclic-fragment of a 1,4:3,6-dianhydrohexitol of structural formula (II), and R⁷ is —(CH₂)₄—.

19. The composition of claim 1, wherein the polymer is a PEUR described by structural formula (V) or (VI).

20. The composition of claim 19, wherein at least one R¹ is a residue of α,ω-bis (4-carboxyphenoxy) (C₁-C₈) alkane, 3,3′-(alkanedioyldioxy)dicinnamic acid, or 4,4′-(alkanedioyldioxy)dicinnamic acid, or at least one R⁴ is a bicyclic-fragment of a 1,4:3,6-dianhydrohexitol of structural formula (II).

21. The composition of claim 19, wherein at least one R¹ is a residue of α,ω-bis (4-carboxyphenoxy) (C₁-C₈) alkane, 3,3′(alkanedioyldioxy)dicinnamic acid or 4,4′(alkanedioyldioxy)dicinnamic acid, or a mixture thereof, and at least one R⁴ is a bicyclic-fragment of a 1,4:3,6-dianhydrohexitol of structural formula (II), and R⁷ is —(CH₂)₄—.

22. The composition of claim 1, wherein the polymer is a PEU described by structural formula (VI) or (VII).

23. The composition of claim 22, wherein at least one R¹ is a bicyclic-fragment of a 1,4:3,6-dianhydrohexitol of structural formula (II) and R⁷ is —(CH₂)₄—.

24. The composition of claim 1, wherein the composition is formulated for administration in the form of a liquid dispersion of the polymer particles.

25. The composition of claim 1, wherein the polymer comprises at least one hydrophilic side chain functional group.

26. The composition of claim 25, wherein the side chain functional group is —COOH.

27. The composition of claim 1, wherein the 1,4:3,6-dianhydrohexitol (II) is derived from D-glucitol, D-mannitol, or L-iditol.

28. The composition of claim 1, wherein the composition forms a time release polymer depot when administered in vivo.

29. The composition of claim 1, wherein the composition biodegrades over a period of about twenty-four hours to about 90 days.

30. The composition of claim 1, wherein the composition is in the form of particles having an average diameter in the range from about 10 nanometers to about 1000 microns.

31. The composition of claim 1, wherein the composition further comprises at least one bioactive agent dispersed in the polymer.

32. The composition of claim 31, wherein at least one bioactive agent is conjugated to the polymer on the exterior of the particles.

33. The composition of claim 31, wherein the bioactive agent is selected from the group consisting of a targeting ligand, a drug, RNA, DNA, an antigen, an antibody, a lipid, and a mono- or polysaccharide.

34. The composition of claim 1, further comprising a covering water soluble molecule conjugated to the polymer on the exterior of the particles.

35. The composition of claim 34, wherein the covering water soluble molecule is selected from the group consisting of poly(ethylene glycol) (PEG); phosphoryl choline (PC); glycosaminoglycans; polysaccharides; poly(ionizable or polar amino acids); chitosan and alginate.

36. The composition of claim 1, wherein a polymer molecule in the particles has an average molecular weight in range from about 5,000 to about 300,000.

37. The composition of claim 1, wherein at least one bioactive agent is conjugated to a polymer molecule in the particles.

38. The composition of claim 1, wherein the composition forms a time release polymer depot when administered in vivo.

39. The composition of claim 1, wherein the particles have an average diameter in the range from about 10 nanometers to about 1000 microns and the at least one bioactive agent is dispersed in the particles.

40. The composition of claim 39, wherein the particles further comprise a covering of the polymer.

41. The composition of claim 1, wherein the composition further comprises a pharmaceutically acceptable vehicle.

42. The composition of claim 1, wherein the composition is in the form of disperse droplets containing the particles in a mist.

43. The composition of claim 42, wherein the mist is produced by a nebulizer.

44. The composition of claim 1, wherein the composition is contained within a nebulizer actuatable to produce a mist comprising dispersed droplets of the particles in a vehicle.

45. The composition of claim 1, wherein the composition is contained within an injection device that is actuatable to administer the composition by injection.

46. The composition of claim 1, wherein the particles encapsulate an aqueous solution containing at least one smaller particle of the polymer in which the at least one macromolecular biologic is dispersed.

47. The composition of claim 1, wherein the particles encapsulate an aqueous solution containing the at least one macromolecular biologic.

48. The composition of claim 1, wherein the composition is formulated for intrapulmonary or gastroenteral delivery.

49. A micelle-forming polymer particle delivery composition comprising at least one macromolecular biologic conjugated via at least one attachment site thereof to a biodegradable polymer comprising
a) a hydrophobic section comprising a biodegradable polymer having a chemical structure described by structural formulas (I) and (III-VII), or a mixture thereof, and
b) a water soluble section comprising:
1) at least one block of ionizable poly(amino acid), or repeating alternating units of polyethylene glycol, polyglycosaminoglycan, or polysaccharide; and
2) at least one ionizable or polar amino acid,
wherein the repeating alternating units have substantially similar molecular weights and wherein the molecular weight of the polymer is in the range from about 10 kD to 300 kD.

50. The composition of claim 49, wherein the molecular weight of the polymer is over 10 kD and at least one of the amino acid units is an ionizable or polar amino acid selected from the group consisting of serine, glutamic acid, aspartic acid, lysine and arginine.

51. The composition of claim 49 wherein the repeating alternating units have substantially similar molecular weights in the range from about 300 D to about 700 D.

52. The composition of claim 49, further comprising a pharmaceutically acceptable aqueous media with a pH value at which at least a portion of the ionizable amino acids in the water soluble chain are ionized, and wherein the composition forms micelles.

53. The composition of claim 49, wherein the micelles have an average size in the range from about 20 nm to about 200 nm.

54. The composition of claim 49, wherein the water soluble section of the polymer has a molecular weight in the range from about 5 kD to about 100 kD.

55. The composition of claim 54, wherein the complete water soluble section of the polymer comprises ionizable or polar water soluble poly(amino acids).

56. The composition of claim 54, wherein the hydrophobic section of the polymer has a chemical structure described by structural formula I, III or VI.

57. The composition of claim 56, wherein the polymer comprises a moiety selected from carboxylate phenoxy propene (CPP), leucine-1,4:3,6-dianhydro-D-sorbitol (DAS), and combinations thereof.

58. The composition of claim 49, wherein the macromolecular biologic is in the form of a protein, polypeptide, polynucleotide, macromolecular lipid, polysaccharide, lipopeptide, lipoprotein, glycopeptide or glycoprotein.

59. The composition of claim 58, wherein the macromolecular biologic is in the form of an oligomer.

60. The composition of claim 3, wherein the oligomer is a sextet of insulin promoters.

61. The composition of claim 49, wherein the macromolecular biologic is in the form of a protein crystal or aggregate.

62. The composition of claim 61, wherein the protein crystal or aggregate further comprises at least one atom of calcium or a transition metal.

63. The composition of claim 61, wherein the protein aggregate is a crystal of insulin oligomers.

64. The composition of claim 63, wherein the crystal of insulin oligomers further comprises at least one zinc atom.

65. The composition of claim 49, wherein the composition is formulated for oral delivery.

66. A method for delivering a macromolecular biologic to a subject comprising administering to the subject in vivo a polymer particle delivery composition of claim 1 in the form of a liquid dispersion of the polymer particles, which particles biodegrade by enzymatic action to release the macromolecular biologic with substantial native activity over time.

67. A method of delivering a macromolecular biologic in vivo with substantial native activity at a controlled rate, said method comprising
1) administering the polymer particles of claim 1 into an in vivo site in the body of the subject, and
2) delivering the macromolecular biologic to the interior body site with substantial native activity and at a controlled rate.

68. The method of claim 67, wherein the particles have an average diameter in the range from about 1 μm to about 200 μm.

69. The method of claim 67, wherein the particles are injected into the interior body site and, agglomerate to form a polymer depot of particles of increased size.

70. The method of claim 69, wherein the composition is administered orally, intramuscularly, subcutaneously, intravenously, into the Central Nervous System (CNS), into the peritoneum or intraorgan.

71. The method of claim 67, wherein macromolecular biologic is human insulin and the administration is orally.
